SEQUENCE LISTING (15 sequences)

SEQ ID NO: 1 - protein, 10 aa, Artificial Sequence
  P5 and stapled-P5 consensus amino acid sequence
  MISC_FEATURE (3)..(3): X is either R or 2,(4, pentenyl)alanine
  MISC_FEATURE (7)..(7): X is either R or 2,(4, pentenyl)alanine
RLXPASXPDT

SEQ ID NO: 2 - protein, 132 aa, Artificial Sequence
  F1 amino acid sequence
SREDFPLIETEDEAYLRRIKMELQQKDKALVELLREKVGLFAEMTHFQVEEDSGGVALPALPRGLFRSESLECPRGERLLQDAIREVEGLKDLLVGPGVELLLTPRDPALLVDPDSGGSTSPGVTANGEARN

SEQ ID NO: 3 - protein, 19 aa, Artificial Sequence
  P5 amino acid sequence
GQEDYDRLRPASYPDTDVI

SEQ ID NO: 4 - protein, 9 aa, Artificial Sequence
  core TAT amino acid sequence
RKKRRQRRR

SEQ ID NO: 5 - protein, 32 aa, Artificial Sequence
  TAT-P5 amino acid sequence
GRKKRRQRRRPWQGQEDYDRLRPASYPDTDVI

SEQ ID NO: 6 - protein, 10 aa, Artificial Sequence
  stapled-P5 amino acid sequence
  MISC_FEATURE (3)..(3): X is 2,(4, pentenyl)alanine
  MISC_FEATURE (7)..(7): X is 2,(4, pentenyl)alanine
RLXPASXPDT

SEQ ID NO: 7 - protein, 164 aa, Artificial Sequence
  TAT-F1 amino acid sequence
MGGYGRKKRRQRRRGGGSGYPYDVPDYAGGSSSREDFPLIETEDEAYLRRIKMELQQKDKALVELLREKVGLFAEMTHFQVEEDSGGVALPALPRGLFRSESLECPRGERLLQDAIREVEGLKDLLVGPGVELLLTPRDPALLVDPDSGGSTSPGVTANGEARN

SEQ ID NO: 8 - protein, 32 aa, Artificial Sequence
  TAT-scrambled P5 amino acid sequence
GRKKRRQRRRPWQDYIAEVPRSDYDLRPTGQD

SEQ ID NO: 9 - protein, 10 aa, Artificial Sequence
  VSV amino acid sequence
TDIEMNRLGK

SEQ ID NO: 10 - DNA, 396 bp, Artificial Sequence
  F1 nucleotide sequence
tccagggaggacttccccctgattgagacagaggatgaggcttacctgcggcgaattaagatggagttgcagcagaaggaccgggcactgtggagctgctgcgagagaagtcggctgttcgctaagatgacccatttccaggccgaagaggctggtggcagtgggctggccctgcccaccatgcccaggggccttttccgctctgagtcccttgagtcccctcgtggcgagcggctgctgcaggatgccatccgtgaggtggagggtctgaaagacctgctggtggggccaggagtggaactgctcttgacaccccgagagccagccctgcccttggaaccagacagcggtggtaacacgagtcctggggtcactgccaatggtgaggccagaaat

SEQ ID NO: 11 - DNA, 57 bp, Artificial Sequence
  P5 nucleotide sequence
ggccaggaggactacgacaggctgaggcccgccagctaccccgacaccgacgtgatc

SEQ ID NO: 12 - protein, 6 aa, Artificial Sequence
  amino acid sequence
GQEDYD

SEQ ID NO: 13 - protein, 16 aa, Artificial Sequence
  P5 and stapled-P5 consensus amino acid sequence
  MISC_FEATURE (9)..(9): X is either R or 2,(4, pentenyl)alanine
  MISC_FEATURE (13)..(13): X is either R or 2,(4, pentenyl)alanine
GQEDYDRLXPASXPDT

SEQ ID NO: 14 - protein, 13 aa, Artificial Sequence
  P5 and stapled-P5 consensus amino acid sequence
  MISC_FEATURE (3)..(3): X is either R or 2,(4, pentenyl)alanine
  MISC_FEATURE (7)..(7): X is either R or 2,(4, pentenyl)alanine
RLXPASXPDTDVI

SEQ ID NO: 15 - protein, 19 aa, Artificial Sequence
  P5 and stapled-P5 consensus amino acid sequence
  MISC_FEATURE (9)..(9): X is either R or 2,(4, pentenyl)alanine
  MISC_FEATURE (13)..(13): X is either R or 2,(4, pentenyl)alanine
GQEDYDRLXPASXPDTDVI

(19) United States
(12) Patent Application Publication
(10) Pub. No.: US 2023/0190869 A1
(43) Pub. Date: Jun. 22, 2023

(54) PEPTIDE INHIBITORS OF GUANINE NUCLEOTIDE EXCHANGE FACTOR H-1

(71) Applicant: UCL Business Ltd, London (GB)

(72) Inventors: Maria S. BALDA, London (GB); Karl MATTER, London (GB)

(21) Appl. No.: 17/794,001

(22) PCT Filed: Jan. 22, 2020
(86) PCT No.: PCT/GB2020/050150

§ 371 Date: Jul. 20, 2022

(51) Int. Cl. A61K 38/17; C07K 14/47; A61P 29/00; A61P 37/08

(52) U.S. Cl. CPC A61K 38/1709; C07K 14/4703; A61P 29/00; A61P 37/08

(57) ABSTRACT

The present invention relates to peptide antagonists or inhibitors of GEF-H1, pharmaceutical compositions comprising said antagonists, polynucleotides encoding said antagonists, vectors encoding said polynucleotides, uses of said antagonists, pharmaceutical compositions and vectors in methods of medical treatment and kits comprising said antagonists, pharmaceutical compositions and vectors. The peptide antagonists of the present invention inhibit RhoA binding to the DH/PH module of GEF-H1 and, thereby, GEF-H1 function

### SEQUENCE LISTING

The instant application contains a Sequence Listing which has been submitted electronically in ASCII format and is hereby incorporated by reference in its entirety. Said ASCII copy, created on Jan. 3, 2023, is named 50318-06300_Sequence_Listing_1_3_23_5T25 and is 7,797 bytes in size.

### FIELD OF THE INVENTION

The present invention relates to peptide antagonists of GEF-H1, pharmaceutical compositions comprising said antagonists, polynucleotides encoding said antagonists, vectors encoding said polynucleotides, uses of said antagonists, pharmaceutical compositions and vectors in methods of medical treatment and kits comprising said antagonists, pharmaceutical compositions and vectors. The peptide antagonists of the present invention inhibit RhoA binding to the DH/PH module of GEF-H1.

### BACKGROUND OF THE INVENTION

Epithelia and endothelia are fundamental for the structure and function of organs and complex tissues. They form cellular barriers that shield our bodies and separate body compartments. Examples of striking importance are the corneal epithelium, which forms a protecting layer at the surface of the eye, the retinal pigment epithelium (RPE), which forms a barrier to the blood at the back of the retina and is important for a functional retina as well as endothelial cells not only in the retina but other parts of our body. Pathological responses involving degeneration, inflammation and malfunction of epithelia and endothelia are major components of many clinically relevant diseases and often lead to loss of organ function. In the eye, many severe diseases such as chronic inflammations, infections, mechanical traumas as well as age-related macular degeneration, diabetes and glaucoma, lead to reduced or loss of vision due to malfunctioning epithelial and endothelial cells. Although such diverse disease conditions are triggered by different stimuli and involve various molecular mechanisms, they share certain fundamental subcellular signalling pathways. One such signalling protein is Guanine Nucleotide Exchange Factor H1 (GEF-H1), a regulator of RhoGTPases involved in fibrotic and inflammatory responses in endothelia as well as epithelia, such as the RPE and the corneal epithelium (Tsapara et al., 2010).

RhoGTPases are important regulatory switches of physiological and pathological pathways that guide processes such as cell migration, gene expression and proliferation. RhoGTPases are regulated by guanine nucleotide exchange factors (GEFs), which activate their signalling functions. In contrast to the GTPases themselves and many of their effectors, which regulate multiple processes in different pathways, Rho GEFs are more process-specific; hence, targeting a specific GEF enables inhibition of specific processes (Terry et al., 2010). GEF-H1 is one such activator that stimulates RhoA and is a crucial effector of a signalling mechanism that drives epithelial dedifferentiation and thereby contributes to multiple pathologies, ranging from inflammatory responses triggered by cytokines and infections to TGFβ-induced dedifferentiation, oncogenic Ras and p53 signalling leading to tumour cell invasion (Mizuarai et al., 2006). Once activated, GEF-H1 drives pathways to altered gene expression and dedifferentiation, cell migration and dissociation of cell junctions. Many adult tissues express little if any GEF-H1 under normal conditions but upregulate its expression due to pathological stimuli. In the RPE GEF-H1 is upregulated in patients in response to mechanical traumas and uveitis. Upregulation occurs in response to TGFβ signalling and mediates α-smooth muscle actin (αSMA) induction, linking GEF-H1 to fibrotic responses. Inhibition or depletion of GEF-H1 function can attenuate pathological processes (induction of αSMA, monolayer contraction, cell migration, proinflammatory signalling, increases in paracellular permeability) without affecting epithelial or endothelial integrity and barrier formation. Prolonged inhibition using siRNA-mediated loss of function approaches does not lead to the induction of compensatory mechanisms.

Current therapies have limiting side effects and, there are currently no solutions existing or under development that target GEF-H1 therapeutically. Inhibitors for Rho signalling components that also contribute to GEF-H1 function exist; however, they lack disease specificity. For example, inhibition of TGFβ with ALK5 inhibitors inhibits many other pathways, and ROCK inhibition has many other effects not related to GEF-H1 function (e.g., ROCK inhibition attenuates epithelial barrier formation). Inhibitors targeting Rho GEFs more generally have also been described but their specificity has not been determined (WO2011/157819). As different Rho GEFs can have opposing functions, specificity for specific GEFs is crucial to block disease-specific pathways.

### SUMMARY OF THE INVENTION

The inventors have investigated the interaction between RhoA and GEF-H1 in order to identify peptide inhibitors or antagonists that could block GEF-H1 function by blocking the interaction between RhoA and GEF-H 1. The inventors have identified several peptides that inhibit GEF-H1 function by blocking RhoA binding to the DH/PH module of GEF-H1. The inventors have shown that GEF-H1 is a promising therapeutic target that is a common component of pathological responses in epithelia and endothelia. GEF-H1 inhibition is able to lead to prolonged downregulation of pathological signalling mechanisms without leading to defects in tissue organisation.

Thus, the invention provides:
A peptide that blocks the function of human Guanine Nucleotide Exchange Factor-H1 (GEF-H1), wherein the peptide comprises an amino acid sequence that has at least 80% sequence identity to SEQ ID NO: 1, SEQ ID NO: 13, SEQ ID NO: 14, SEQ ID NO: 15, or SEQ ID NO: 2.

The invention also provides:
A polynucleotide sequence encoding a peptide described herein, or a vector comprising said polynucleotide.

The invention also provides:
A pharmaceutical composition comprising a peptide described herein or a vector as described herein, and a pharmaceutically acceptable carrier.

The invention also provides:
A peptide described herein, or a vector described herein, or a pharmaceutical composition described herein, for use in therapy.

The invention also provides:
A peptide described herein, or a vector described herein, or a pharmaceutical composition described herein for use in a method of treating an inflammatory or degenerative disease, or cancer.

The invention also provides:
A method of treating or preventing a disease, such as an inflammatory or degenerative disease, or cancer comprising administering a therapeutically effective amount of a peptide described herein, or a pharmaceutical composition described herein, or a vector described herein to a patient in need thereof.

The invention also provides:
The use of a peptide described herein, or a vector described herein, or a pharmaceutical composition described herein, in the manufacture of a medicament to treat an inflammatory or degenerative disease, or cancer.

The invention also provides:
A kit comprising a peptide, vector, or pharmaceutical composition described herein, and instructions for use.

### BRIEF DESCRIPTION OF THE SEQUENCES

SEQ ID NOs: 1, 13, 14, and 15—P5 and stapled-P5 consensus amino acid sequence.

SEQ ID NO: 2 Fragment 1 (F1) amino acid sequence.

SEQ ID NO: 3—Peptide 5 (P5) amino acid sequence.

SEQ ID NO: 4—core TAT amino acid sequence.

SEQ ID NO: 5—TAT-P5 amino acid sequence.

SEQ ID NO: 6—Stapled-P5 amino acid sequence.

SEQ ID NO: 7—TAT-F1 amino acid sequence.

SEQ ID NO: 8—TAT-scrambled P5 amino acid sequence.

SEQ ID NO: 9—VSV amino acid sequence.

SEQ ID NO: 10—F1 nucleotide sequence.

SEQ ID NO: 11—P5 nucleotide sequence.

SEQ ID NO: 12—GQEDYD.

### BRIEF DESCRIPTION OF THE DRAWINGS

FIG. 1—GEF-H1 is expressed in human corneal epithelial (HCE) cells and regulates NFkB activity.

(A, B) Expression of GEF-H1: HCE cells were (A) stained for GEF-H1 and the junctional marker ZO-1 or (B) transfected with control or GEF-H1-specific siRNAs and analysed by immunoblotting as indicated. (C) Activity of GEF-H1 in low confluent, proliferating HCE cells was analysed by pulldown with GST-RhoA. Note, pulldown indicates GEF-H1 is active in low confluent HCE cells. (D) Cells were transfected with siRNAs as in (B) but then analysed by staining for junctional proteins. Note, GEF-H1 depleted cells still form intact monolayers. (E, F) NFkB activity was assayed by luciferase reporter gene assay in GEF-H1-depleted cells (E) or in cells transfected with the C-terminal domain (CTD) of GEF-H1 (F). The CTD of GEF-H1 blocks proinflammatory signalling.

FIG. 2—Mapping of GEF-H1 C-terminal domain peptide fragments

(A) Domain structure of GEF-H1. Indicated are the C1 domain, the Dbl homology domain (DH), and Pleckstrin homology domain (PH), and the C-terminal auto-inhibitory domain (CTD). Removal of the C1 domain and CTD domain leads to a constitutive active mutant (GEF-H1-CA) as the C1 domain mediates sequestration of GEF-H1 to microtubules and the CTD contains an auto-inhibitory activity.

(B) The inhibitory domain was divided and the fragments generated were assayed for inhibitory activity.

(C) Identification of the inhibitory sequence Fragment 1 (F1) and derivatives constructed for functional assays to inhibit GEF-H1.

FIG. 3—Identification of GEF-H1 peptide inhibitors

(A) Structural modelling of the DH/PH module of GEF-H1 with RhoA. The sequences corresponding to the DH/PH module of GEF-H1 were fitted into the existing crystal structures of RhoA-bound to other Dbl family guanine nucleotide exchange factors. The resulting interaction surface of GEF-H1 with RhoA was used to select sequences of RhoA likely to be important for binding of RhoA to GEF-H 1.

(B) Inhibitors derived from the structural model and experimentally verified. Also shown is the TAT-scrambled P5 (negative control) sequence GRKKRRQRRRPWQDYIAEVPRSDYDLRPTGQD (SEQ ID NO: 8).

FIG. 4—Mapping of GEF-H1 auto-inhibitory region and functional GEF-H1 inhibitors

(A, B) Stable cell lines with tetracycline (Tet) inducible expression of HA-tagged GEF-H1 were transiently transfected with plasmids encoding VSV-tagged CTD fragments of GEF-H1 (see FIG. 2) or (B) by peptide transfection to test inhibitors identified from the modelling of the RhoA/GEF-H1 interaction (see FIG. 3). Cell areas were then quantified to measure attenuation of GEF-H1-induced cell spreading (C, CTD fragments; D, peptides). (E) SRE (serum response element) promoter activity was assayed with a luciferase reporter gene assay to determine inhibition of GEF-H1 by CTD fragments. *p<0.05; **p>0.01; t-test.

FIG. 5—GEF-H1 inhibitors block binding of RhoA

Recombinant fusion proteins of GST and RhoA were used for pulldown of constitutively active GEF-H1 from cell extracts. (A) Candidate peptide inhibitors were added to the pulldown. (B, C) Recombinant fragments from the CTD of GEF-H1 were added. Note, the further analysed P5 and F1 peptide inhibitors both block the interaction between RhoA and active GEF-H1.

FIG. 6—Functionality of membrane-permeable GEF-H1 inhibitors

Inhibitors rescue GEF-H1 overexpression changes in cell shape and transcription. (A) TAT-F1 (4 μM) or (B) TAT-P5 (20 μM) rescue GEF-H1 overexpression-induced cell shape changes by >70%. (C) TAT-F1 or (D) TAT-P5 inhibit transcription of the SRE-containing alpha-smooth muscle actin (αSMA) promoter.

FIG. 7—Specificity of membrane-permeable GEF-H1 inhibitors

Inhibitors do not block signalling by related Dbl family GEFs. (A) MDCK cells conditionally expressing p114RhoGEF, a close homologue of GEF-H1, were treated with tetracycline to induce VSV-tagged p114RhoGEF expression and were treated with TAT-F1 (4 μM) or TAT-P5 (20 μM) before and during the induction, prior to fixation and staining for F-actin, the tight junction protein ZO-1, and the induced transgene (VSV). Note, p114RhoGEF overexpression induces cell rounding, which is not inhibited by either inhibitor. (B) TAT-F1 (4 μM) or TAT-P5 (20 μM) do not inhibit transcription from the αSMA promoter in cells expressing overexpressing active Dbl.

FIG. 8—TAT-F1 and TAT-P5 are not cytotoxic

Inhibitors do not induce apoptosis or necrosis. (A and B) MDCK and HEK293 cells were incubated as described in previous figures with TAT-F1 or TAT-P5. Cell toxicity was then assessed by measuring release of LDH in the medium and caspase 3/7 activity in cell extracts. No significant cell toxicity was induced at the different concentrations analysed for TAT-F1 (1-4 μM), TAT-P5 (5-20 μM). Direct lysis of cells served as a positive control in (A) whereas active caspases were used as controls in (B).

FIG. 9—Functionality and specificity of stapled-P5

Stapled-P5 inhibits GEF-H1 signalling. (A) Transfected stapled-P5 (stP5) inhibits GEF-H1-induced cell shape changes in MDCK cells (B) Stapled-P5 added to the medium inhibited constitutively active GEF-H1-induced signalling in transfected 293T cells assessed by αSMA and SRE-reporter assays. (C) Stapled-P5 did not inhibit cell shape changes in MDCK cells induced by p114RhoGEF (D) Stapled-P5 did not inhibit transcription in the αSMA and SRE-reporter assays in cells overexpressing active Dbl.

FIG. 10—Stapled-P5 is not cytotoxic

Inhibitor does not induce apoptosis or necrosis. (A and B) MDCK and HEK293 cells were incubated as described in previous figures with with stapled-P5. Cell toxicity was then assessed by measuring release of LDH in the medium and caspase 3/7 activity in cell extracts. No significant cell toxicity was induced at the different concentrations analysed. Direct lysis of cells served as a positive control for LDH release whereas active caspases were used in caspase assays.

FIG. 11—GEF-H1 inhibitors block processes associated with epithelial degeneration

Inhibition of TGF-β signaling in primary retinal pigment epithelial (RPE) cells. (A, B) Primary RPE cells were incubated with TAT-P5 or TAT-F1 and stimulated with TGF-β for five days. (A) αSMA and, as a loading control, tubulin expression were analysed by immunoblotting. The graph shows a quantification of αSMA expression normalised by tubulin expression derived from densitometric scanning of the immunoblots. Note, the concentration-dependent repression of TGF-β induced αSMA expression. (B) Expression of the junctional marker ZO-1 and tubulin were determined. (C) Primary RPE cells were incubated with TGF-β as in (A) but with stapled-P5, and expression of αSMA positive cells was analysed by immunofluorescence.

FIG. 12—TAT-P5 blocks cell migration and degeneration of human stem cell-derived RPE cells

Inhibition of TGF-β signaling in RPE cells. (A) RPE cells were incubated with TAT-P5 or control inhibitor and migration was assayed with and without TGF-β stimulation using a scratch wound assay. (B) Human RPE cells generated from embryonic stem cells were treated with TGF-β and TAT-P5 as indicated and analysed by immunofluorescence for αSMA, the junctional markers ZO-1, cingulin, and GEF-H1. Note, the inhibitor prevents TGF-β-induced junctional disruption and αSMA induction.

FIG. 13—Inflammation: GEF-H1 inhibitors prevent endothelial activation by LPS

GEF-H1 inhibitors prevent endothelial inflammatory responses induced by LPS. Human primary endothelial cells were stimulated with LPS in the presence or absence of the indicated GEF-H1 inhibitors. Cells were then analysed by immunofluorescence (A); immunoblotting for expression of ICAM, a protein upregulated by inflammatory stimuli, and loading controls (B); or by analysing paracellular permeability (C). TAT-P5 and TAT-F1 attenuate cytoskeletal remodelling, ICAM1 induction, and attenuate loss of barrier function. GEF-H1 inhibitors also prevented reorganisation of the cytoskeleton and junction disruption induced by TNFα and thrombin (not shown). Stapled-P5 also inhibited LPS-induced response in endothelia (not shown).

FIG. 14—GEF-H1 inhibitors attenuate proinflammatory signalling

Human corneal epithelial cells were assayed for NF-κB activity using a luciferase reporter assay. After transfection of the reporter plasmids, the cells were incubated with peptide inhibitors as indicated prior to analysis.

FIG. 15—GEF-H1 inhibitors attenuate migration of tumour cells

The invasive breast cancer cell line MDA-MB-231 was cultured on extracellular matrix coated tissue culture plates. Migration was analysed by live cell microscopy during 4 hours to determine the effect of GEF-H1 inhibitors stapled-P5 and TAT-P5. Note, the inhibitors reduced velocity and distance of migration.

FIG. 16—GEF-H1 inhibitors decrease autoimmune response in vivo.

GEF-H1 inhibitors in experimental autoimmune-induced uveoretinitis (EAU). EAU was induced in mice and the GEF-H1 inhibitors were tested topically (daily) and intraperitoneally/subcutaneously (every two days) as soon as inflammation was detected by bright field ocular microscopy. Ocular inflammation in mice started at day 14. Disease progression was followed by endoscopic fundus imaging for 2-3 weeks after treatment started. Control (dots) indicate the calculated clinical scores of EAU disease progression from day 14 to 29 obtained in 7 independent experiments (Control, n=11; stapled P5, n=9; TAT-P5, n=9).

FIG. 17—Attenuation of retinal damage in EAU

(A) Fundus images of IRBP immunized mice treated with PBS or TAT-P5. (B) Histological analysis of control and TAT-P5 treated eyes.

FIG. 18—GEF-H1 inhibitors block allergic conjunctivitis

Inhibition of allergic conjunctivitis in a mouse model. OVA sensitized mice were challenged with OVA in the presence of absence of TAT-P5 GEF-H1 inhibitor applied as eye drops. Images are derived from one mouse, left eye control treatment and right eye TAT-P5 eye drops. (A) Of four mice treated with TAT-P5, the inhibitor treated eye showed a strong attenuation of pathological response in all animals. (B) Periodic Acid-Schiff staining indicating strong reduction of inflammatory cell accumulation in TAT-P5 treated eyes.

### DETAILED DESCRIPTION OF THE INVENTION

It is to be understood that different applications of the disclosed antagonists and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “antagonist” includes “antagonists”, and the like. The terms “antagonist” and “inhibitor” are used interchangeably herein.

All publications, patents and patent applications cited herein, whether supra or infra, are hereby incorporated by reference in their entirety.

Blocking GEF-H1 Activity and/or Function

The peptide antagonists of the present invention inhibit GEF-H1 by blocking binding of RhoA to GEF-H1. The peptide antagonists of the present invention inhibit GEF-H1 by blocking binding of RhoA to the DH/PH module GEF-H1. The peptide antagonists of the present invention may specifically inhibit GEF-H1 by blocking binding of RhoA to the catalytic domain of GEF-H1. The term “specifically inhibits GEF-H1” can be defined as meaning that an antagonist binds to GEF-H1 with greater affinity than to another target and thereby blocks the interaction between GEF-H1 and its substrate RhoA. A peptide antagonist of the invention is preferably capable of inhibiting its target with an affinity that is at least two-fold, 10-fold, 50-fold, 100-fold or greater than for binding to another non-target molecule. The non-target molecule may be another member of the GEF protein family. Specificity of binding can be measured by any suitable means, such as pulldown experiments combined with western blotting to monitor inhibition of GEF-H1/RhoA complex formation.

The peptide antagonists of the present invention block the function and/or activity of GEF-H1. The peptide antagonists of the present invention specifically bind to GEF-H1 to block the function and/or activity of GEF-H1. Blocking of GEF-H1 function and/or activity encompasses any reduction in its activity or function that results in reduced degeneration, inflammation and malfunction of epithelia and endothelia. The peptide inhibitors of the present invention block GEF-H1 function and/or activity by blocking the interaction of GEF-H1 with RhoA. Blocking GEF-H1 function and/or activity can also encompass the inhibition of TGFβ, or inflammatory signalling. Blocking of GEF-H1 function may be via blocking the GEF-H1-mediated activation of RhoA. The peptide antagonists of the present invention bind to GEF-H1 to block the function of GEF-H1. Such an antagonist may be specific to the blocking of the function and/or activity of GEF-H1, that is, it may reduce the function and/or activity of GEF-H1, but not that other related proteins, such as other GEF proteins.

Blocking encompasses both total and partial reduction of GEF-H1 activity or function, for example total or partial prevention of the GEF-H1-RhoA interaction. For example, a blocking antagonist of the invention may reduce the activity of GEF-H1 by from 10 to 50%, at least 50% or at least 70%, 80%, 90%, 95% or 99%.

Blocking of GEF-H1 activity or function can be measured by any suitable means. For example, blocking of GEF-H1 activity or function can be determined by measuring the effect on its interaction with RhoA, by the inhibition of TGFβ-signalling and/or the inhibition of GEF-H1-regulated promoters (e.g., αSMA promoter). Blocking of GEF-H1 activity or function can also be measured via assays that are known to the person skilled in the art. Blocking of GEF-H1 activity or function can also be measured using a biochemical assay to measure inhibition of RhoA binding to GEF-H1.

Blocking may take place via any suitable mechanism, depending for example on the nature (see below) of the antagonist used, e.g. steric interference in any direct or indirect GEF-H1-target interaction.

The peptide antagonist of the present invention may act specifically to block the function of GEF-H1. That is, the effect of the antagonist on GEF-H1 may be greater than any other biological effect of the antagonist. Such an antagonist may be specific to the inhibition of GEF-H1, that is it may decrease the activity of GEF-H1 but not other GEFs.

A peptide antagonist of the present invention may be an antagonist of GEF-H1 as described herein, that does not act as an antagonist of other GEFs. A peptide antagonist of the present invention may act on GEF-H1 in preference to other GEFs. For example, a peptide antagonist of GEF-H1 of the present invention may have one or more of the characteristics of a GEF-H1 antagonist as described herein, but may not have such characteristics in relation to other GEFs, or may have such characteristics to a lower level in relation to other GEFs when compared to GEF-H1. For example, an antagonist that decreases the activity of GEF-H1 may not decrease the activity of other GEFs, or may decrease the activity of other GEFs to a lesser extent, such as a lower percentage decrease, than its effect on GEF-H1. A GEF-H1 peptide antagonist as described herein may have an effect on other GEFs, such as antagonism of the activity or signalling of one or more other GEFs, that is less than 25%, less than 20%, less than 15%, less than 10%, less than 5%, less than 1%, or less than 0.1% the effect of that antagonist on the activity or signalling of GEF-H1.

The specificity of the GEF-H1 peptide antagonist may apply within the whole body of an individual to be treated with the peptide antagonist of the invention, that is the actions of the GEF-H1 peptide antagonist may be specific as discussed above throughout the body of the individual. The specificity of the GEF-H1 peptide antagonist may apply within particular tissues of the individual, such as the eye. The GEF-H1 peptide antagonist may therefore be a specific antagonist of GEF-H1 as described above. For example, the GEF-H1 peptide antagonist may not be an antagonist of other GEFs, or may have no significant effect on the activity of other GEFs.

Peptide Antagonists of GEF-H1

The peptide antagonists of the present invention inhibit GEF-H1 function by blocking RhoA binding to the DH/PH module of GEF-H1, which is the structural unit of GEF-H1 responsible for binding RhoA and catalysing RhoA activation. The peptide antagonists of the present invention inhibit GEF-H1 function by inhibiting the binding of RhoA to the DH/PH module of GEF-H1. Thus, the peptide antagonists of the present invention share a mechanism of action. The peptide antagonist embodiments of the present invention are described in more detail below.

In an embodiment of the present invention peptide antagonists of GEF-H1 are based on fragment of the C-terminal domain (CTD) of GEF-H1. The CTD of GEF-H1 has an auto-inhibitory function and can attenuate epithelial degeneration in an RPE disease model. The auto-inhibitory activity was mapped by the present inventors to a specific amino acid sequence, termed Fragment 1 (F1), that blocked GEF-H1 function either by transfection of a recombinant protein or by fusion to a TAT sequence to render it membrane-permeable. The F1 peptide inhibits binding of RhoA to the DH/PH module of GEF-H1.

The F1 peptide embodiment of the present invention has the following sequence:

(SEQ ID NO: 2)
SREDFPLIETEDEAYLRRIKMELQQKDKALVELLREKVGLFAEMTHFQV
EEDSGGVALPALPRGLFRSESLECPRGERLLQDAIREVEGLKDLLVGPG
VELLLTPRDPALLVDPDSGGSTSPGVTANGEARN.

In a connected embodiment of the present invention, peptide antagonists of GEF-H1 were generated by modelling of the GEF-H1/RhoA complex, with peptides designed that were likely to inhibit formation of the complex. This resulted in the identification of Peptide 5 (P5), a 19 amino acid inhibitory peptide that was retained for further analysis and refinement. The P5 peptide inhibits binding of RhoA to the DH/PH module of GEF-H 1.

The P5 peptide embodiment of the present invention has the following sequence:

(SEQ ID NO: 3)
GQEDYDRLRPASYPDTDVI.

Modifications to the Peptide Antagonists of the Present Invention

The peptide antagonist embodiments of the invention may include, or be bound to, or be conjugated to, additional moieties, or other sequences or elements, such as localisation tags, affinity tags or cell permeability tags. The peptide antagonist embodiments of the invention may include a sequence, such as an arginine-rich amino acid sequence to render them membrane-permeable. The peptide antagonist embodiments of the invention may include or be conjugated to sequences, such as a TAT sequence, to render them membrane-permeable, and/or a VSV sequence, to allow detection with an antibody.

The minimally required core TAT sequence is RKKRRQRRR (SEQ ID NO: 4). Other amino acids can be put in as spacers and to avoid steric problems. In addition, several alternative cell penetrating peptides are also encompassed by the present invention. The biochemical properties of these peptides are generally very similar as the basic clusters (the K and R amino acids) are required for membrane penetration. Exemplary cell-penetrating tags that can be conjugated to the peptide antagonists of the present invention include Penetratin, Trasportan, MPG KALA and Polyarginines such as the R8 or R9 sequences.

In an embodiment of the invention, the peptide antagonist comprises SEQ ID NO: 2 and SEQ ID NO: 4. In a preferred embodiment of the invention, the peptide antagonist of the invention can be a TAT-F1 peptide. An exemplary TAT-F1 peptide of the present invention has the following sequence:

(SEQ ID NO: 7)
MGGYGRKKRRQRRRGGGSGYPYDVPDYAGGSSSREDFPLIETEDEAYLR
RIKMELQQKDKALVELLREKVGLFAEMTHFQVEEDSGGVALPALPRGLF
RSESLECPRGERLLQDAIREVEGLKDLLVGPGVELLLTPRDPALLVDPD
SGGSTSPGVTANGEARN.

In another embodiment of the invention, the peptide antagonist comprises SEQ ID NO: 3 and SEQ ID NO: 4. In a related embodiment of the invention, derivatives of the P5 peptide antagonist of the invention were also synthesized: TAT-P5, a variant with a TAT motif to promote entry into cells, and stapled-P5, a shortened variant with an intramolecular link to stabilise the conformation.

In an embodiment of the invention, the antagonist of the invention can be a TAT-P5 peptide. An exemplary TAT-P5 peptide of the present invention can have the following sequence:

(SEQ ID NO: 5)
GRKKRRQRRRPWQGQEDYDRLRPASYPDTDVI.

In an embodiment of the invention, the antagonist of the invention can be a P5 peptide, a TAT-P5 peptide or a stapled-P5 peptide. The stapled-P5 peptide of the present invention can be defined according to the following formula:

(SEQ ID NO: 6)
RL(X)PAS(X)PDT; X = 2,(4, pentenyl)alanine
(which forms the staple)).

A consensus sequence comprising the stapled-P5 and P5 peptide embodiments of the invention can be written as below:

(SEQ ID NOs: 1, 13, 14, and 15)
X₁RLX₂PASX₃PDTX₄

### Wherein

X₁ is either omitted or an amino acid sequence consisting of GQEDYD (SEQ ID NO: 12);
X₂ is either R or 2,(4, pentenyl)alanine;
X₃ is either R or 2,(4, pentenyl)alanine, with the proviso that when X₂ is 2,(4, pentenyl)alanine X₃ is 2,(4, pentenyl)alanine, and when X₃ is 2,(4, pentenyl)alanine X₂ is 2,(4, pentenyl)alanine; and
X₄ is either omitted or an amino acid sequence consisting of DVI.

Thus, in an embodiment of the invention, the peptide antagonist comprises or consists of SEQ ID NO: 1, SEQ ID NO: 13, SEQ ID NO: 14, or SEQ ID NO: 15.

In an embodiment of the invention, the peptide antagonists of the invention are conjugated to an epitope tag such as a VSV sequence. In an embodiment of the invention, the VSV sequence is TDIEMNRLGK (SEQ ID NO: 9). In an embodiment of the invention, the peptide antagonists of the invention comprise SEQ ID NO: 1, SEQ ID NO: 13, SEQ ID NO: 14, or SEQ ID NO: 15, and SEQ ID NO: 9, SEQ ID NO: 2 and SEQ ID NO: 9, or SEQ ID NO: 3 and SEQ ID NO: 9.

The present invention also encompasses peptide variants of the peptide antagonists described herein such as the F1, TAT-F1, P5, TAT-P5 and stapled-P5 peptides, that retain the ability to block the function of GEF-H1. Blocking the function of GEF-H1 is defined as above. Peptidomimetics may also be designed to mimic such blocking peptides.

A peptide variant of the F1 peptide may comprise at least 100, 105, 110, 115, 120, 125 or 130 consecutive amino acids from SEQ ID NO: 2, whilst maintaining the activity of the F1 peptide described herein. A peptide variant of the F1 peptide may comprise at least 100, 105, 110, 115, 120, 125 or 130 consecutive amino acids from SEQ ID NO: 2 and be no more than 132 amino acids in length, whilst maintaining the activity of the F1 peptide described herein. A peptide variant of the F1 peptide may consist of 100, 105, 110, 115, 120, 125 or 130 consecutive amino acids from SEQ ID NO: 2, whilst maintaining the activity of the F1 peptide described herein.

A peptide variant of the P5 peptide may comprise at least 10, 11, 12, 13, 14, 15, 16, 17, 18 or 19 consecutive amino acids from SEQ ID NO: 3, whilst maintaining the activity of the P5 peptide described herein. A peptide variant of the P5 peptide may comprise at least 10, 11, 12, 13, 14, 15, 16, 17, 18 or 19 consecutive amino acids from SEQ ID NO: 3 and be no more than 19 amino acids in length, whilst maintaining the activity of the P5 peptide described herein. A peptide variant of the P5 peptide may consist of 10, 11, 12, 13, 14, 15, 16, 17, 18 or 19 consecutive amino acids from SEQ ID NO: 3, whilst maintaining the activity of the P5 peptide described herein.

A peptide variant of the F1 peptide or TAT-F1 peptide embodiments of the invention may comprise 1, 2, 3, 4, 5, up to 10, up to 20, up to 30, up to 40, up to 50 or more amino acid substitutions and/or deletions from the specific sequences and fragments discussed above, whilst maintaining the activity of the peptides described herein. A peptide variant of the P5 peptide embodiment of the present invention, including the TAT-P5 or stapled P5 variants, may comprise 1, 2, 3, 4, 5, up to 10 or more amino acid substitutions and/or deletions from the specific sequences and fragments discussed above, whilst maintaining the activity of the peptides described herein.

“Deletion” variants may comprise the deletion of, for example, 1, 2, 3, 4 or 5 individual amino acids or of one or more small groups of amino acids such as 2, 3, 4 or 5 amino acids. “Small groups of amino acids” can be defined as being sequential, or in close proximity but not sequential, to each other. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids, which can be used to select suitable substituents, are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged |
| | | | (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, |
| | | | neutral |
| Lys | polar, hydrophilic, charged(+) | Trp | aromatic, hydrophobic, |
| | | | neutral |
| Leu | aliphatic, hydrophobic, neutral | Tyr | aromatic, polar, hydrophobic |

Preferred variants include those in which instead of the naturally occurring amino acid, the amino acid which appears in the sequence is a structural analog thereof. Amino acids used in the sequences may also be derivatized or modified, e.g. labelled, providing the function of the peptide is not significantly adversely affected.

Preferably variant peptides according to the present invention have an amino acid sequence which has more than 60%, or more than 70%, e.g. 75 or 80%, preferably more than 85%, e.g. more than 90, 95%, 96%, 97%, 98% or 99% amino acid identity to the peptides disclosed herein. This level of amino acid identity may be seen across the full-length of the relevant SEQ ID NO sequence or over a part of the sequence, such as across 5, 10 or 15 or more amino acids of SEQ ID NOs: 3 or 5; or 5, 10, 15, 20, 30, 40, 50, 100 or more amino acids of SEQ ID NOs: 2 or 7 depending on the size of the full-length peptide.

Thus, the invention encompasses a peptide that comprises an amino acid sequence that has more than 60%, or more than 70%, e.g. 75 or 80%, preferably more than 85%, e.g. more than 90, 95%, 96%, 97%, 98% or 99% sequence identity to the F1 peptide (SEQ ID NO: 2), TAT-F1 peptide (SEQ ID NO: 7), P5 peptide (SEQ ID NO: 3), TAT-P5 peptide (SEQ ID NO: 5) or stapled P5 peptide (RL(X)PAS(X)PDT; X=2,(4, pentenyl)alanine (which forms the staple)) (SEQ ID NO: 6) or the P5 consensus sequence (see SEQ ID NO: 1, SEQ ID NO: 13, SEQ ID NO: 14, and SEQ ID NO: 15), wherein the peptide blocks the function of GEF-H1.

Thus, the invention encompasses a peptide that comprises the sequence of the F1 peptide (SEQ ID NO: 2), TAT-F1 peptide (SEQ ID NO: 7), P5 peptide (SEQ ID NO: 3), TAT-P5 peptide (SEQ ID NO: 5), stapled P5 peptide (RL(X)PAS(X)PDT; X=2,(4, pentenyl)alanine (which forms the staple)) (SEQ ID NO: 6) or the P5 consensus sequence (see SEQ ID NO: 1, SEQ ID NO: 13, SEQ ID NO: 14, and SEQ ID NO: 15). Thus, the invention encompasses a peptide that has, or consists of, the sequence of the F1 peptide (SEQ ID NO: 2), TAT-F1 peptide (SEQ ID NO: 7), P5 peptide (SEQ ID NO: 3), TAT-P5 peptide (SEQ ID NO: 5), stapled P5 peptide (RL(X)PAS(X)PDT; X=2,(4, pentenyl)alanine (which forms the staple)) (SEQ ID NO: 6) or the P5 consensus sequence (see SEQ ID NO: 1, SEQ ID NO: 13, SEQ ID NO: 14, and SEQ ID NO: 15). The term “has”, can be used interchangeably with the term “consists of”.

In connection with amino acid sequences, “sequence identity” refers to sequences, which have the stated value when assessed using ClustalW (Thompson et al., 1994) with the following parameters:

Pairwise alignment parameters—Method: accurate, Matrix: PAM, Gap open penalty: 10.00, Gap extension penalty: 0.10;

Multiple alignment parameters -Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: G, P, S, N, D, Q, E, K, R. Sequence identity at a particular residue is intended to include identical residues which have simply been derivatized.

In particular embodiments, the peptide antagonists of the present invention may be linked (directly or indirectly) to another moiety. The other moiety may be a therapeutic agent such as a drug. The other moiety may be a detectable label. The other moiety may be a binding moiety specific for a therapeutic target. The therapeutic agent or detectable label may be directly attached, for example by chemical conjugation.

Polynucleotides, Vectors and Host Cells

The present invention also encompasses polynucleotides, vectors and expression vectors encoding the peptides described herein.

The invention also relates to polynucleotides that encode peptides of the invention. Thus, a polynucleotide of the invention may encode any peptide as described herein. The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, genomic DNA, recombinant polynucleotides, plasmids, vectors, isolated DNA of any sequence, isolated RNA of any sequence, nucleic acid probes, and primers. A polynucleotide of the invention may be provided in isolated or purified form.

A nucleic acid sequence which “encodes” a selected polypeptide is a nucleic acid molecule, which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence. The polynucleotides of the invention can be modified as most amino acids are encoded by more than one codon. The polynucleotides of the invention can be modified in view of codon degeneracy in order to encode a peptide of the invention.

In one embodiment, a polynucleotide of the invention comprises a sequence which encodes a F1 peptide or variant amino acid sequence as described above. In one embodiment, a polynucleotide of the invention comprises the sequence of SEQ ID NO: 10, which encode a F1 peptide as described above.

SEQ ID NO: 10 (F1 nucleotide sequence):
TCCAGGGAGGACTTCCCCCTGATTGAGACAGAGGATGAGGCTTACCTGCG
GCGAATTAAGATGGAGTTGCAGCAGAAGGACCGGGCACTGTGGAGCTGCT
GCGAGAGAAGTCGGCTGTTCGCTAAGATGACCCATTTCCAGGCCGAAGAG
GCTGGTGGCAGTGGGCTGGCCCTGCCCACCATGCCCAGGGGCCTTTTCCG
CTCTGAGTCCCTTGAGTCCCCTCGTGGCGAGCGGCTGCTGCAGGATGCCAT
CCGTGAGGTGGAGGGTCTGAAAGACCTGCTGGTGGGGCCAGGAGTGGAA
CTGCTCTTGACACCCCGAGAGCCAGCCCTGCCCTTGGAACCAGACAGCGG
TGGTAACACGAGTCCTGGGGTCACTGCCAATGGTGAGGCCAGAAAT.

In an alternative embodiment a polynucleotide of the invention comprises a sequence which encodes a P5 peptide or variant amino acid sequence as described above. In one embodiment, a polynucleotide of the invention comprises the sequence of SEQ ID NO: 11, which encodes a P5 peptide as described above.

SEQ ID NO: 11 (P5 nucleotide sequence):
GGCCAGGAGGACTACGACAGGCTGAGGCCCGCCAGCTACCCCGACACCG
ACGTGATC.

Variant polynucleotide sequences are also encompassed by the present invention. Such variants may have a sequence having at least 70%, at least 80%, at least 90%, at least 95%, at least 96%, at least 97%, at least 98% or at least 99% sequence identity to a polynucleotide sequence that encodes a peptide described herein, wherein the peptide encoded by the variant polynucleotide retains the functionality of a peptide described herein.

A peptide of the invention may thus be produced from or delivered in the form of a polynucleotide, which encodes, and is capable of expressing it.

Polynucleotides of the invention can be synthesised according to methods well known in the art, as described by way of example in Sambrook et al. (1989, Molecular Cloning—a laboratory manual; Cold Spring Harbor Press).

The polynucleotides of the present invention may be provided in the form of an expression cassette, which includes control sequences operably linked to the inserted sequence, thus allowing for expression of the peptide of the invention in vivo. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. These expression cassettes, in turn, are typically provided within vectors (e.g., plasmids or recombinant viral vectors). Such an expression cassette may be administered directly to a host subject.

Alternatively, a vector comprising a polynucleotide of the invention may be administered to a host subject. Preferably the polynucleotide is prepared and/or administered using a genetic vector. A suitable vector may be any vector, which is capable of carrying a sufficient amount of genetic information, and allowing expression of a peptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals, which may be necessary, and which are positioned in the correct orientation, in order to allow for expression of a peptide of the invention. Other suitable vectors would be apparent to persons skilled in the art. By way of further example in this regard we refer to Sambrook et al. (1989, Molecular Cloning—a laboratory manual; Cold Spring Harbor Press).

A person skilled in the art may use the amino acid sequences described herein to clone or generate cDNA or genomic sequences. Cloning of these sequences in an appropriate eukaryotic expression vector, like pcDNA3 (Invitrogen), or derivates thereof, and subsequent transfection of mammalian cells (like CHO cells) will result in the expression and secretion of the peptides described herein.

The invention also includes cells that have been modified to express a peptide of the invention. Such cells include transient, or preferably stable higher eukaryotic cell lines, such as mammalian cells or insect cells, lower eukaryotic cells, such as yeast or prokaryotic cells, such as bacterial cells. Particular examples of cells, which may be modified by insertion of vectors or expression cassettes encoding for a peptide of the invention, include mammalian HEK293, CHO, HeLa, NS0 and COS cells.

Such cell lines of the invention may be cultured using routine methods to produce a peptide of the invention, or may be used therapeutically or prophylactically to deliver peptides thereof of the invention to a subject. Alternatively, polynucleotides, expression cassettes or vectors of the invention may be administered to a cell from a subject ex vivo and the cell then returned to the body of the subject.

The present invention encompasses expression constructs comprising a polynucleotide sequence encoding an F1 peptide or variant thereof, or a P5 peptide or variant thereof, wherein the variant maintains the ability to block GEF-H1 function or activity. An expression construct may be defined as a polynucleotide sequence capable of driving protein expression from a polynucleotide sequence containing a coding sequence.

The expression constructs of the present invention comprise promoters and a polynucleotide sequence encoding an F1 peptide or variant thereof, wherein the variant maintains the ability to block GEF-H1 function or activity. The F1 peptide sequence encoded by the polynucleotide sequence is preferably that of SEQ ID Nos: 2 or 7. The P5 peptide sequence encoded by the polynucleotide sequence is preferably that of SEQ ID NO: 1, 13, 14, 15, 3, or 5. The expression construct can comprise any promoter sequence suitable to express the F1 or P5 peptide-encoding polynucleotide sequences in the cells of interest. The promoter may be a ubiquitous promoter. The promoter may be a cell-specific promoter. The promoter may be specific to the cells of the eye.

The present invention provides vectors comprising the expression constructs of the present invention. The vector may be of any type, for example it may be a plasmid vector or a minicircle DNA.

Typically, vectors of the invention are however viral vectors. The viral vector may be based on the herpes simplex virus, adenovirus or lentivirus. The viral vector may be an adeno-associated virus (AAV) vector or a derivative thereof.

The viral vector derivative may be a chimeric, shuffled or capsid modified derivative.

The viral vector may comprise an AAV genome from a naturally derived serotype, isolate or clade of AAV.

The serotype may for example be AAV2, AAV5 or AAV8.

The vector of the present invention may comprise an adeno-associated virus (AAV) genome or a derivative thereof.

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly and with the additional removal of the AAV rep and cap genes, the AAV genome of the vector of the invention is replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype or isolate or clade of AAV. As is known to the skilled person, AAV viruses occurring in nature may be classified according to various biological systems.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, recently identified from primate brain. In vectors of the invention, the genome may be derived from any AAV serotype. The capsid may also be derived from any AAV serotype. The genome and the capsid may be derived from the same serotype or different serotypes.

In vectors of the invention, it is preferred that the genome is derived from AAV serotype 2 (AAV2), AAV serotype 4 (AAV4), AAV serotype 5 (AAV5) or AAV serotype 8 (AAV8). It is most preferred that the genome is derived from AAV2 but other serotypes of particular interest for use in the invention include AAV4, AAV5 and AAV8.

The present invention thus encompasses the following embodiments:
An expression construct comprising in a 5′ to 3′ direction: (a) a promoter polynucleotide sequence; and
   (b) a polynucleotide sequence that encodes a sequence as shown in SEQ ID nOs: 2 or 7, or a sequence having at least 90% sequence identity to SEQ ID nOs: 2 or 7 that retains the functionality of the F1 peptide.
An expression construct comprising in a 5′ to 3′ direction: (a) a promoter polynucleotide sequence; and
   (b) the polynucleotide sequence of SEQ ID NO: 10, or a sequence having at least 90% sequence identity to SEQ ID NO: 10 that retains the functionality of the F1 peptide.
An expression construct comprising in a 5′ to 3′ direction: (a) a promoter polynucleotide sequence; and
   (b) a polynucleotide sequence that encodes a sequence as shown in SEQ ID nOs: 1, 3 or 5, or a sequence having at least 90% sequence identity to SEQ ID nOs: 1, 3 or 5 that retains the functionality of the P5 peptide.
An expression construct comprising in a 5′ to 3′ direction: (a) a promoter polynucleotide sequence; and
   (b) the polynucleotide sequence of SEQ ID NO: 11, or a sequence having at least 90% sequence identity to SEQ ID NO: 11 that retains the functionality of the P5 peptide.
   Retaining the functionality of the F1 peptide or the P5 peptide can be defined as maintaining the ability of the F1 peptide or P5 peptide to block GEF-H1 activity or function.
A vector comprising an expression construct described herein.
A vector comprising an expression construct described herein, wherein the vector is a viral vector.
A viral vector comprising an expression construct described herein, wherein the vector is an adeno-associated virus (AAV) vector or comprises an AAV genome or a derivative thereof.
A host cell that contains a vector described herein or produces a viral vector described herein.
A pharmaceutical composition comprising a vector as described herein and a pharmaceutically acceptable carrier.
The vector or pharmaceutical composition described herein for use in a method of preventing or treating a disease described herein, such as an inflammatory or degenerative disease, or cancer.

Pharmaceutical Compositions, Dosages and Dosage Regimes

The peptide antagonists of the present invention or polynucleotides of the present invention, or vectors of the present invention may be part of a composition. Compositions of the invention will typically be formulated into pharmaceutical compositions, together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular, subcutaneous, intraocular or intravitreal administration (e.g., by injection or infusion). Depending on the route of administration, the composition may be coated in a material to protect the composition from the action of acids and other natural conditions that may inactivate the composition.

In certain embodiments, a pharmaceutically acceptable carrier comprises at least one carrier selected from the group consisting of a co-solvent solution, liposomes, micelles, liquid crystals, nanocrystals, nanoparticles emulsions, microparticles, microspheres, nanospheres, nanocapsules, polymers or polymeric carriers, surfactants, suspending agents, complexing agents such as cyclodextrins or adsorbing molecules such as albumin, surface active particles, and chelating agents. In further embodiments, a polysaccharide comprises hyaluronic acid and derivatives thereof, dextran and derivatives thereof, cellulose and derivatives thereof (e.g. methylcellulose, hydroxy-propylcellulose, hydroxy-propylmethylcellulose, carboxymethylcellulose, cellulose acetate phthalate, cellulose acetate succinate, cellulose acetate butyrate, hydroxypropylmethyl-cellulose phthalate), chitosan and derivative thereof, [beta]-glucan, arabinoxylans, carrageenans, pectin, glycogen, fucoidan, chondrotin, dermatan, heparan, heparin, pentosan, keratan, alginate, cyclodextrins, and salts and derivatives, including esters and sulfates, thereof.

Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be preferable to include isotonic agents, for example, sugars, trehalose, polyalcohols such as mannitol, sorbitol, or sodium chloride in the composition

The pharmaceutical compositions of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating the active agent in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Pharmaceutical compositions of the invention may comprise additional active ingredients as well as an antagonist of the invention. Compositions of the invention may comprise one or more antagonists of the invention. They may also comprise additional therapeutic or prophylactic active agents.

In a preferred embodiment, the pharmaceutical composition according to the invention is in a form selected from the group consisting of an aqueous solution, a gel, a hydrogel, a film, a paste, a cream, a spray, an ointment, or a wrap. In further embodiments, the pharmaceutical compositions described herein can be administered by a route such as intravenous, subcutaneous, intraocular, intramuscular, intra-articular, intradermal, intraperitoneal, spinal or by other parenteral routes of administration, for example by injection or infusion. Administration may be rectal, oral, ocular, topical, epidermal or by the mucosal route. Administration may be local, including peritumoral, juxtatumoral, intratumoral, to the resection margin of tumors, intralesional, perilesional, by intra cavity infusion, intravesicle administration, or by inhalation. In a preferred embodiment, the pharmaceutical composition is administered intravenously or subcutaneously. In a preferred embodiment, the pharmaceutical composition is administered intraocularly or via eye drops.

The compositions of the present invention may be administered for prophylactic and/or therapeutic treatments. In therapeutic applications, compositions are administered to a subject already suffering from a disorder or condition as described herein, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

In prophylactic applications, compositions are administered to a subject at risk of a disorder or condition as described herein, in an amount sufficient to prevent or reduce the subsequent effects of the condition or one or more of its symptoms. An amount adequate to accomplish this is defined as a “prophylactically effective amount”. Effective amounts for each purpose will depend on the severity of the disease or injury as well as the weight and general state of the subject. An example of a condition that may be treated prophylactically in the context of the invention is wet AMD (age-related macular degeneration); one eye may develop the condition before the other, with the first eye being treated once the problem is recognised and the second prophylactically.

A subject for administration of the compositions of the invention may be a human or non-human animal. The term “non-human animal” includes all vertebrates, e.g., mammals and non-mammals, such as non-human primates, sheep, dogs, cats, horses, cows, chickens, amphibians, reptiles, etc. Administration to humans is preferred.

The compositions of the present invention may be administered via one or more routes of administration using one or more of a variety of methods known in the art. As will be appreciated by the skilled artisan, the route and/or mode of administration will vary depending upon the desired results. Preferred routes of administration for compositions of the invention include intravenous, subcutaneous, intraocular, intramuscular, intradermal, intraperitoneal, spinal or other parenteral routes of administration, for example by injection or infusion. The phrase “parenteral administration” as used herein means modes of administration other than enteral and topical administration, usually by injection. Administration may be rectal, oral, ocular, topical, epidermal or by the mucosal route. Administration may be local, including peritumoral, juxtatumoral, intratumoral, to the resection margin of tumors, intralesional, perilesional, by intra cavity infusion, intravesicle administration, or by inhalation. In a preferred embodiment, the pharmaceutical composition is administered intravenously or subcutaneously. In a preferred embodiment, the pharmaceutical composition is administered intraocularly or via eye drops.

A suitable dosage of the composition of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.1 μg/kg to about 10 mg/kg body weight of the patient to be treated. For example, a suitable dosage may be from about 1 μg/kg to about 10 mg/kg body weight per day. For intraocular administration, a suitable dosage may be from about 1 to 5 μg or 1 to 10 μg. For subcutaneous or intraperitoneal administration, a suitable dosage may be from about 0.5 to 5 mg/kg or from about 0.5 to 10 mg/kg.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration may be in single or multiple doses. Multiple doses may be administered via the same or different routes and to the same or different locations. Alternatively, doses can be via a sustained release formulation, in which case less frequent administration is required. Dosage and frequency may vary depending on the half-life of the antagonist in the patient and the duration of treatment desired.

Diseases to be Treated

The peptide antagonists of the present invention, the pharmaceutical compositions of the present invention, and the vectors of the present invention can be used in therapy and the treatment of disease.

The peptide antagonists of the present invention can specifically block GEF-H1 signalling in vitro and block epithelial and endothelial degeneration in cell-based models of degenerative and inflammatory disease. The peptide antagonists of the invention also block other GEF-H1 functions, such as TGFβ-driven cell migration (Tsapara et al., 2010) and tumour cell migration. Two distinct in vivo models further support the concept of using GEF-H1 inhibitors to treat inflammatory and degenerative diseases.

Thus, the peptide antagonists of the present invention can be used to treat diseases that are associated with GEF-H1 signalling. Thus, the peptide antagonists of the present invention can be used to treat inflammatory and degenerative diseases. Based on the properties of the inhibitors and the known functions of GEF-H1, the identified peptides can be applied to the treatment of different types of diseases.

GEF-H1 regulates inflammatory signalling, epithelial and endothelial barrier integrity, migration, and gene expression leading during fibrotic disease. GEF-H1 also regulates immune cell activation. Hence, the peptide antagonists of the present invention can be used to treat inflammatory and degenerative diseases such as:
allergic and inflammatory disease of the eye (corneal inflammation, conjunctivitis) and other organs such the lung (asthma, chronic obstructive pulmonary disease) (Mambetsariev et al., 2014; Birukova et al., 2010);
autoimmune diseases such as uveoretinitis, multiple sclerosis, rheumatoid arthritis;
fibrotic diseases, such as those leading to tissue contraction in the eye (proliferative vitreoretinopathy, choroidal neovascularization, glaucoma and trabecular meshwork fibrosis) or fibrotic diseases that affect other organs such as the lung (idiopathic pulmonary fibrosis), kidney or liver (Tsapara et al., 2010);
diabetes, and diabetic complications leading to tissue barrier failures, such as diabetic retinopathy and diabetic nephropathy;
neurological degenerative diseases, such as Huntington disease, and Alzheimer's disease and other tauopathies (Varma et al., 2010).

GEF-H1 is a driver of metastatic and invasive cell migration, this includes cancers that are still difficult to treat such as hepatocellular carcinoma and pancreatic cancer. GEF-H1 is an amplifier of oncogenic Ras signalling and is induced by mutant p53, leading to enhanced proliferation of tumour cells due to its function as regulating RhoA-stimulated proliferation. Mutations in Ras and p53 are very common in many cancers from different tissues. GEF-H1 inhibitors attenuate migration of metastatic tumour cells in culture. Thus, the peptide antagonists of the present invention can also be used in the treatment of cancer. The peptide antagonists of the present invention can also be used in the treatment of metastatic cancer. The peptide antagonists of the present invention can also be used in the treatment of hepatocellular carcinoma and pancreatic cancer.

The peptide antagonists of the present invention can be used in a method of treating an inflammatory or degenerative disease, or cancer.

The peptide antagonists of the present invention, the pharmaceutical compositions of the present invention, and the vectors of the present invention can be used in a method of treating an inflammatory or degenerative disease, or cancer, wherein the inflammatory or degenerative disease is an allergic and inflammatory disease of the eye or other organ such as the lung; an autoimmune disease; a fibrotic disease; a fibrotic disease of the eye or lung, kidney or liver; diabetes; or a neurological degenerative disease.

The peptide antagonists of the present invention, the pharmaceutical compositions of the present invention, and the vectors of the present invention can be used in a method of treating an inflammatory or degenerative disease, or cancer, wherein the inflammatory or degenerative disease to be treated is corneal inflammation, conjunctivitis, asthma, chronic obstructive pulmonary disease, uveoretinitis, multiple sclerosis, rheumatoid arthritis, proliferative vitreoretinopathy, choroidal neovascularization fibrosis, glaucoma, trabecular meshwork fibrosis, idiopathic pulmonary fibrosis, diabetic retinopathy, diabetic nephropathy, Huntington disease, Alzheimer's disease, or wherein the cancer is metastatic cancer.

The peptide antagonists of the present invention, the pharmaceutical compositions of the present invention, and the vectors of the present invention can be administered by any reasonable route. Preferred routes of administration for the peptide antagonists of the invention include intravenous, subcutaneous, intraocular, intramuscular, intradermal, intraperitoneal, spinal or other parenteral routes of administration, for example by injection or infusion. The phrase “parenteral administration” as used herein means modes of administration other than enteral and topical administration, usually by injection. Administration may be rectal, oral, ocular, topical, epidermal or by the mucosal route. Administration may be local, including peritumoral, juxtatumoral, intratumoral, to the resection margin of tumors, intralesional, perilesional, by intra cavity infusion, intravesicle administration, or by inhalation. In a preferred embodiment, the antagonist is administered intravenously or subcutaneously. In a preferred embodiment, the antagonist is administered intraocularly or via eye drops.

The peptide antagonists of the present invention, the pharmaceutical compositions of the present invention, and the vectors of the present invention can be administered in combination with an additional therapeutic agent.

The present invention encompasses a method of treatment of disease comprising administering a therapeutically effective amount of the peptide antagonist of the invention, the pharmaceutical compositions of the present invention or the vectors of the present invention to a patient in need thereof.

The present invention encompasses a method of treating an inflammatory or degenerative disease, or cancer, comprising administering a therapeutically effective amount of a peptide antagonist of the invention, the pharmaceutical compositions of the present invention or the vectors of the present invention, to a patient in need thereof.

The present invention encompasses the use of a peptide antagonist of the invention, the pharmaceutical compositions of the present invention or the vectors of the present invention in the manufacture of a medicament to treat a disease.

The present invention encompasses the use of a peptide antagonist of the invention, the pharmaceutical compositions of the present invention or the vectors of the present invention in the manufacture of a medicament to treat an inflammatory or degenerative disease, or cancer.

The present invention encompasses a pharmaceutical composition comprising the peptide antagonists of the present invention, the pharmaceutical compositions of the present invention or the vectors of the present invention, for the treatment of disease.

The present invention encompasses a pharmaceutical composition comprising the antagonists of GEF-H1 disclosed herein, for the treatment of inflammatory or degenerative disease, or cancer.

Combination Therapies

The peptide antagonists of the present invention, the pharmaceutical compositions of the present invention, and the vectors of the present invention can be used in combination with other therapeutic agents to treat disease.

Kits

Also within the scope of the present invention are kits comprising the peptide antagonists of the present invention, the pharmaceutical compositions of the present invention and/or the vectors of the of the present invention. The kits may contain instructions for use. The kit may further contain one or more additional reagents, such as an additional therapeutic or prophylactic agent as discussed above. The peptide antagonists could be also used individually or in combination in the kits of the invention. The kits of the invention may be used to characterise RhoA signalling pathways in biomedical research.

The following Examples illustrate the invention which should not be construed as further limiting.

### EXAMPLES

### Example 1—Action of GEF-H1

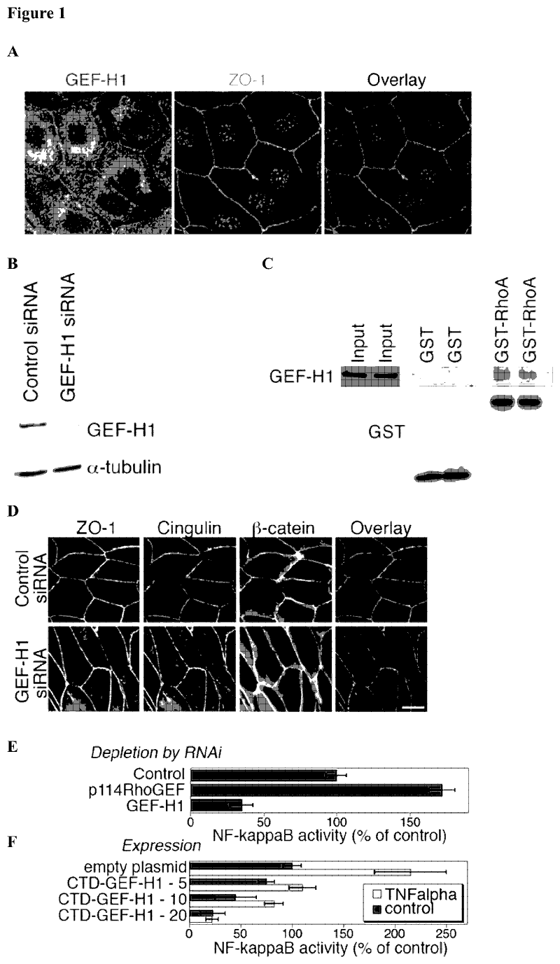

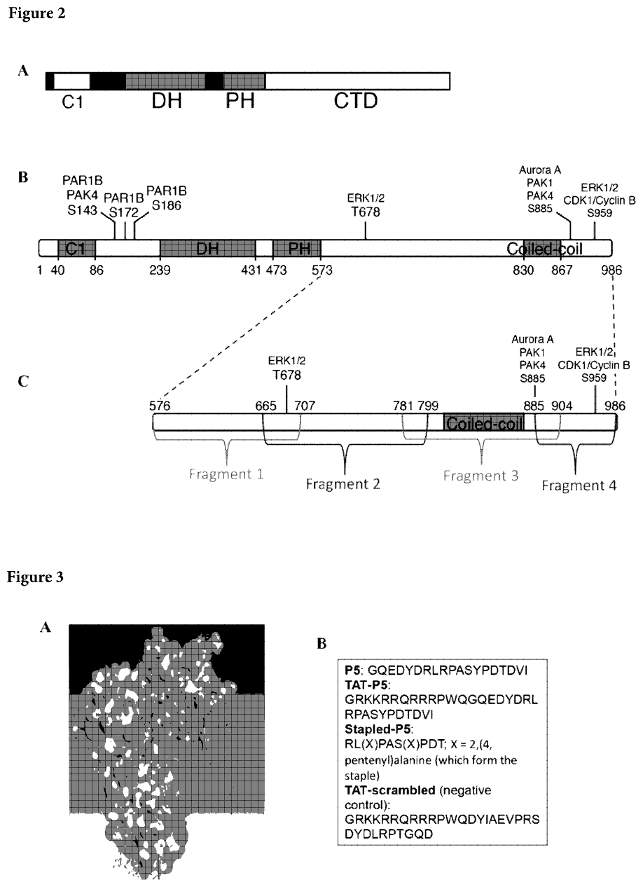

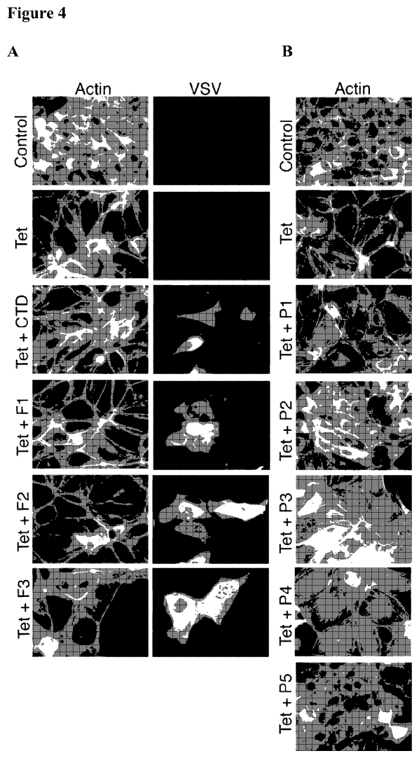

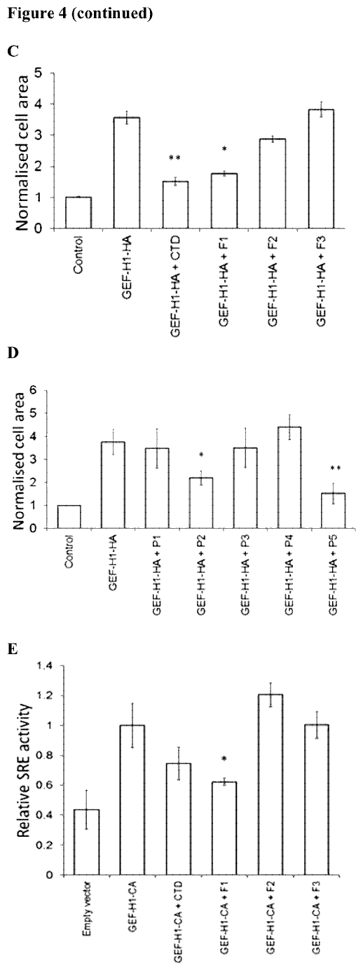

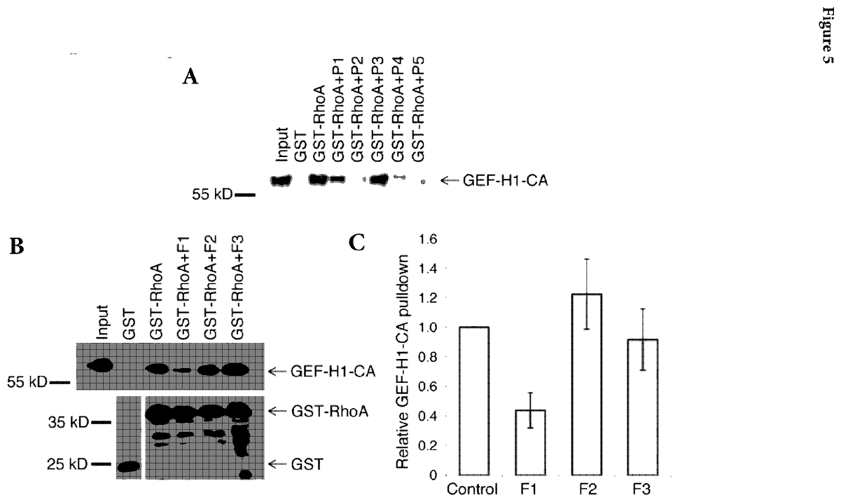

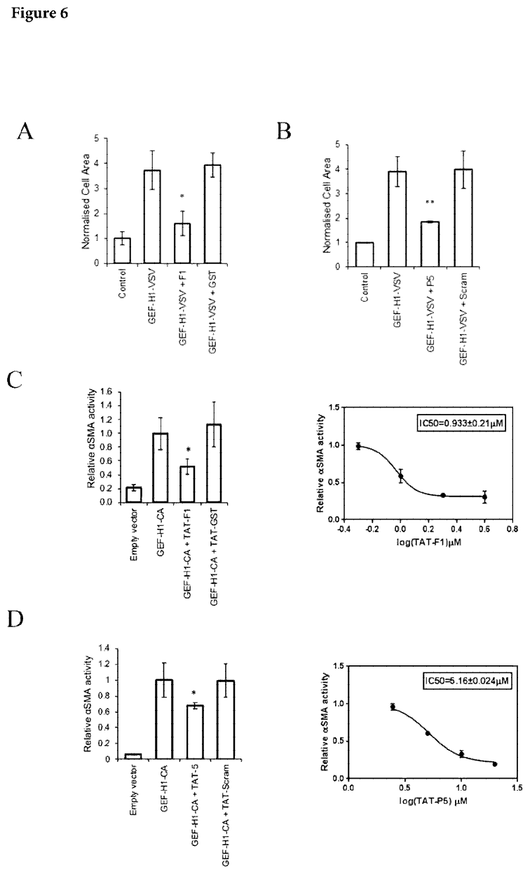

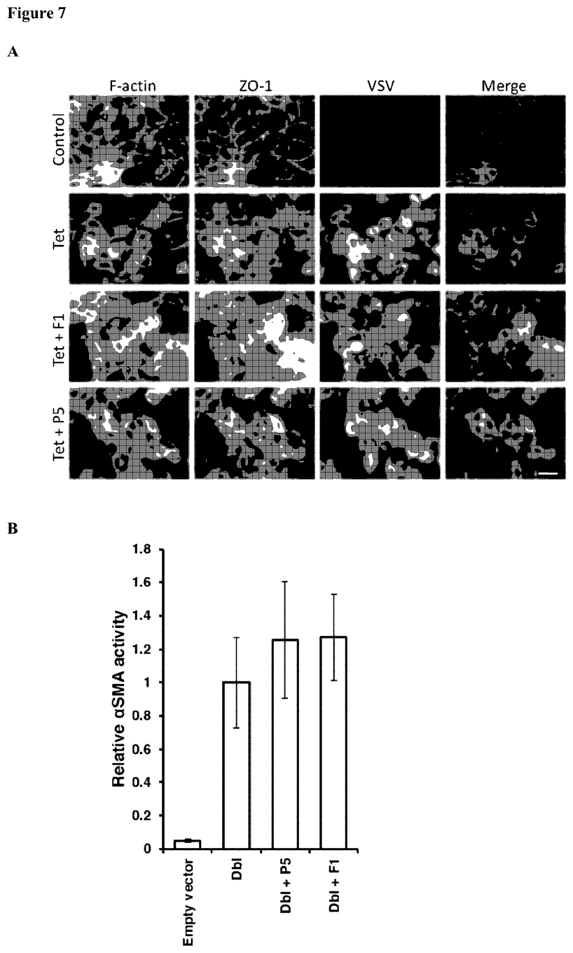

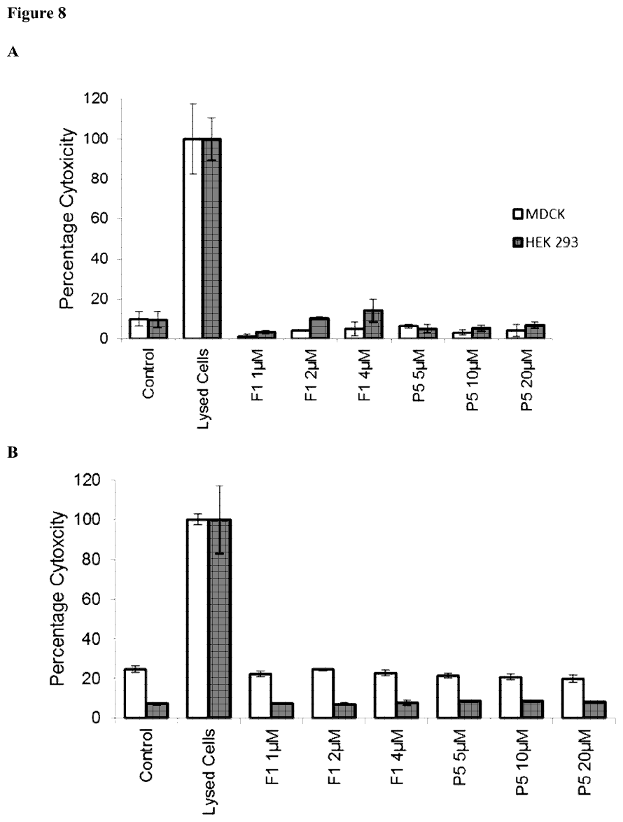

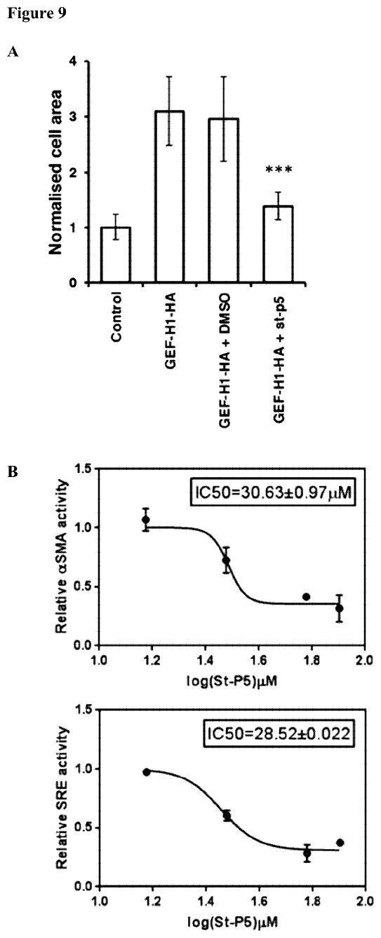

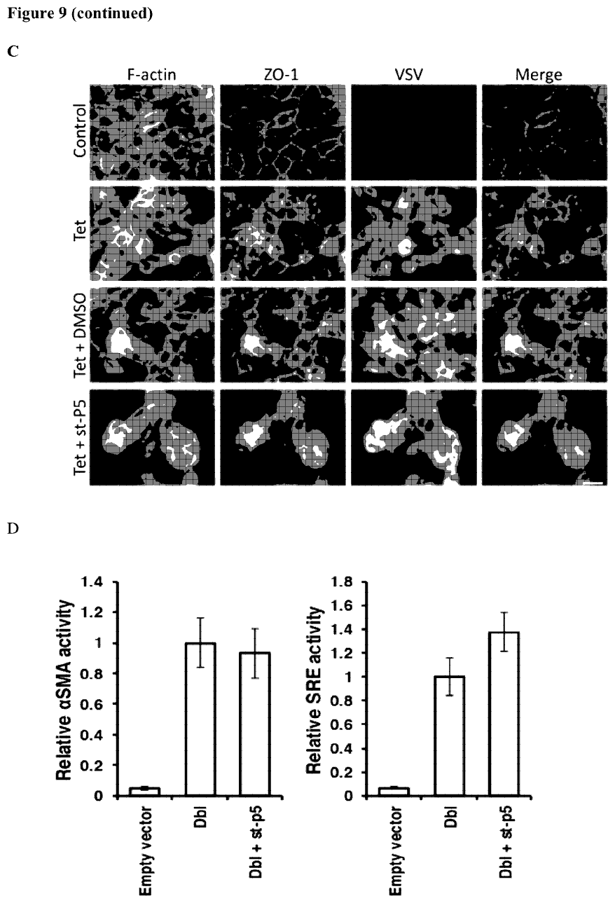

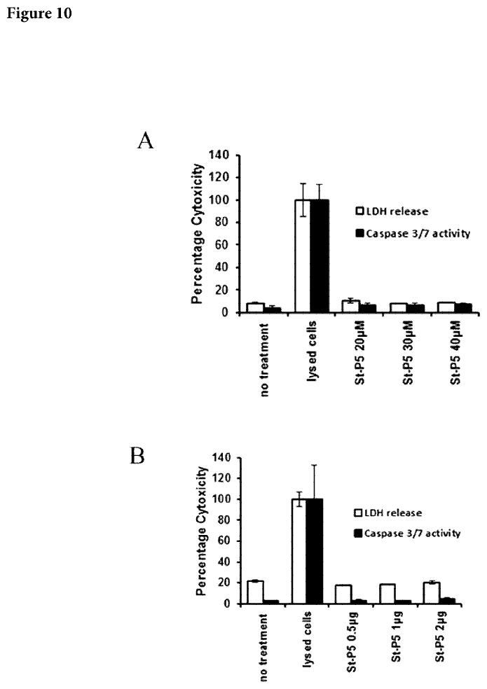

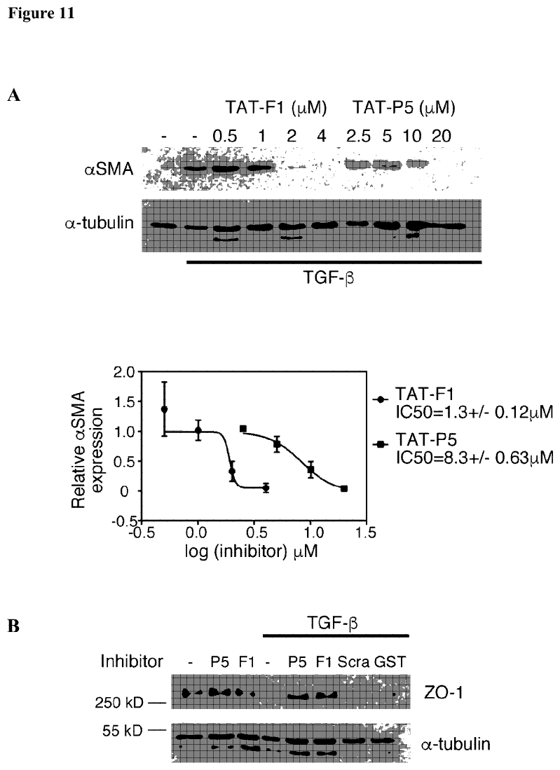

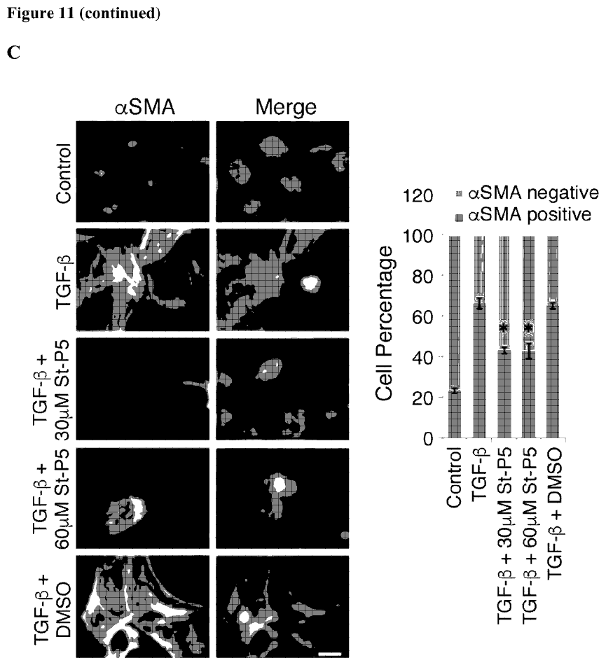

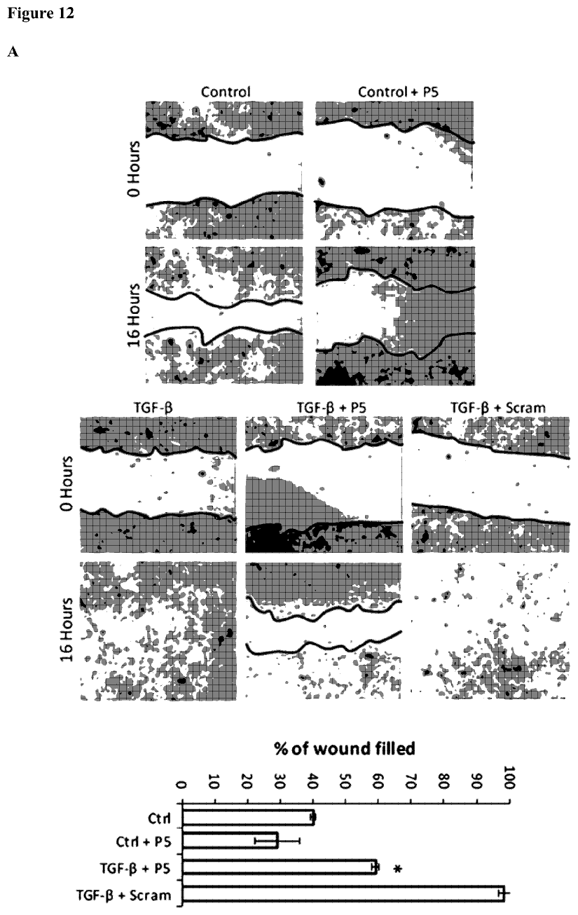

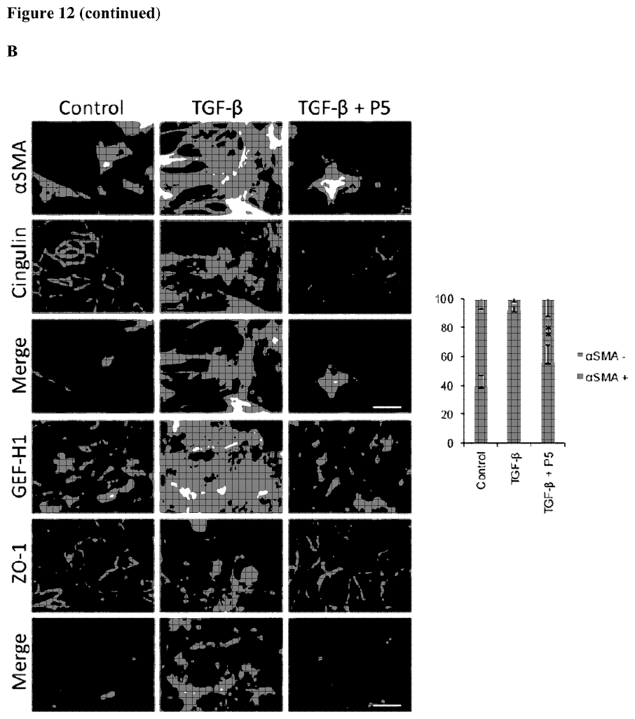

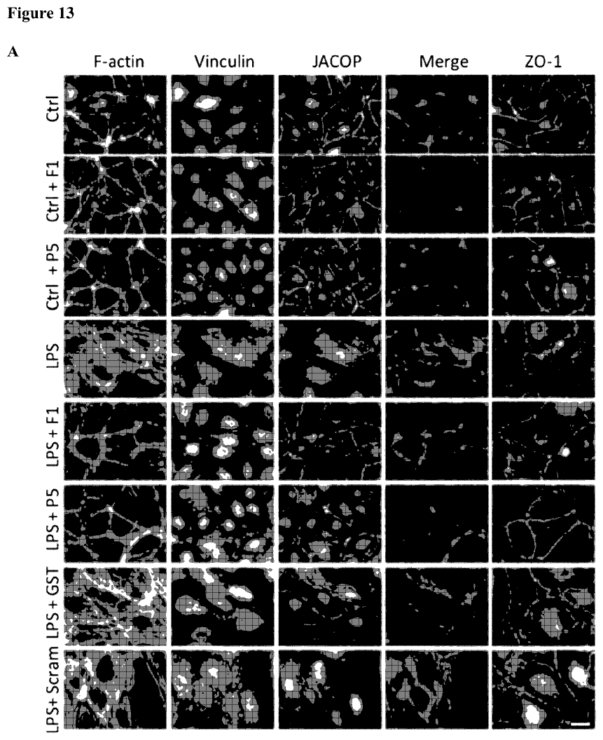

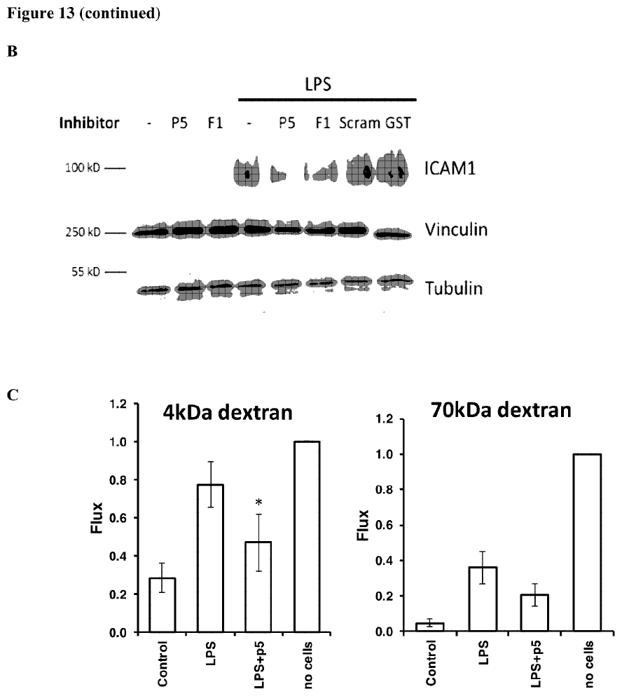

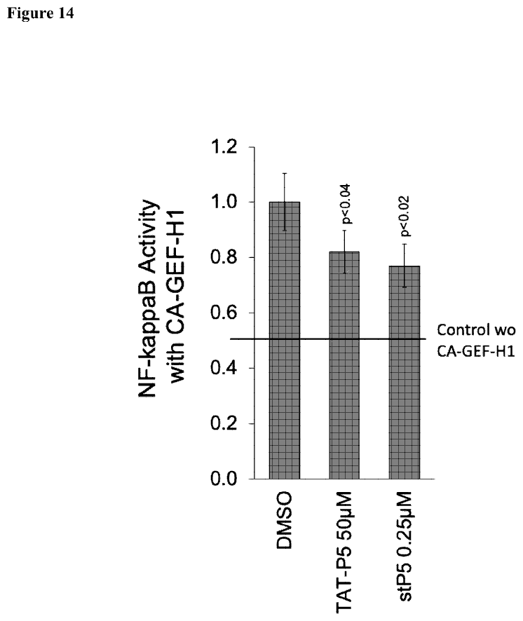

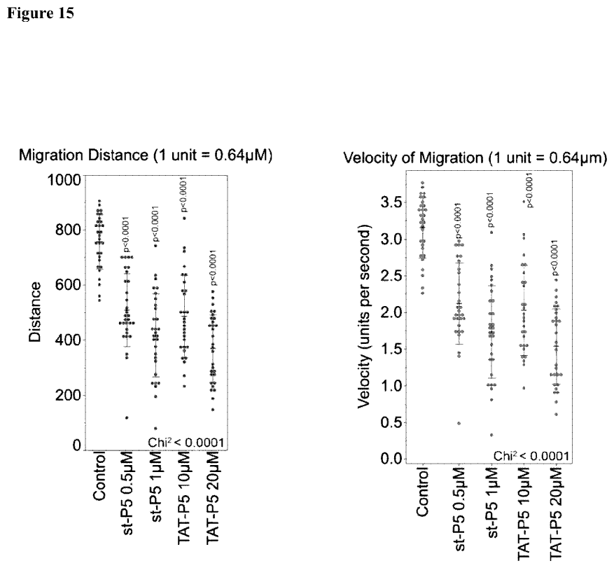

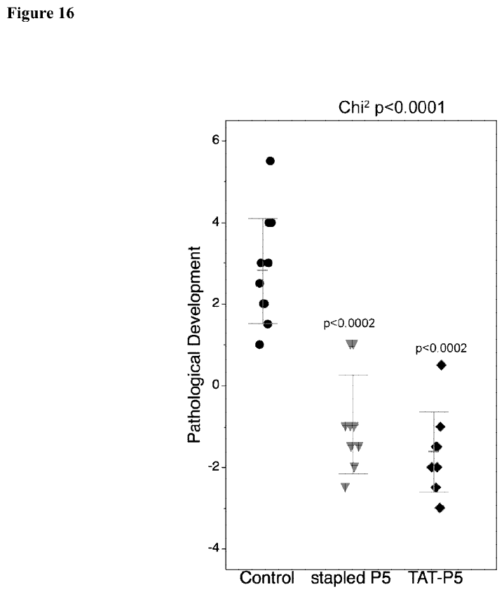

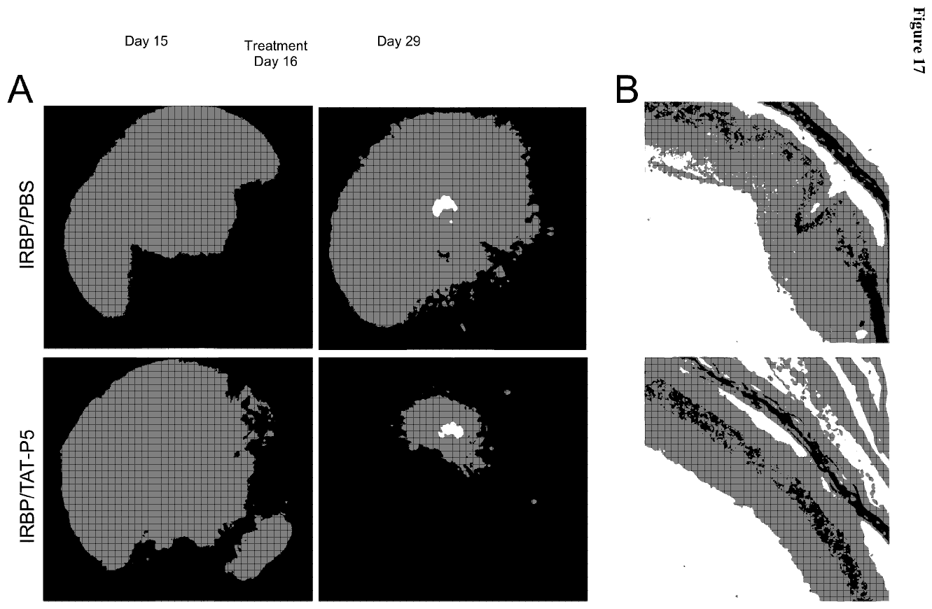

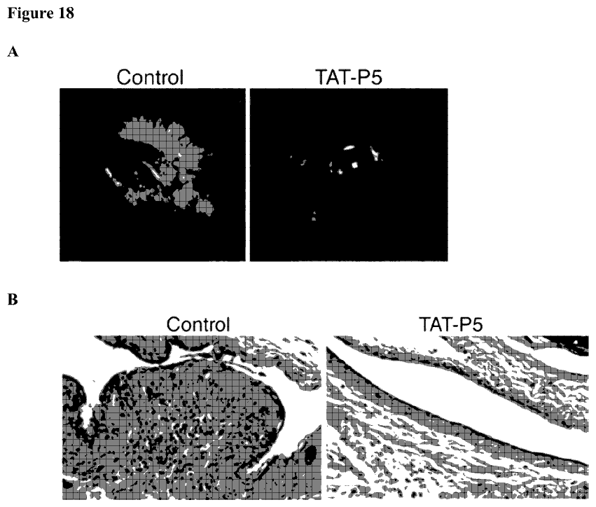

For human corneal epithelial cells (HCE), the data of the inventors show that GEF-H1 regulates proinflammatory signalling such as NFκB activity in response to TNFα, that serum and TNFα stimulated activity can be blocked by depletion or expression of a dominant negative GEF-H1 construct, but that GEF-H1 is not required to maintain monolayer integrity (FIG. 1). HCE cells were (A) stained for GEF-H1 and the junctional marker ZO-1 or (B) transfected with control or GEF-H1-specific siRNAs and analysed by immunoblotting as indicated. (C) Activity of GEF-H1 in low confluent, proliferating HCE cells was analysed by pulldown with GST-RhoA. Note, pulldown indicates GEF-H1 is active in low confluent HCE cells. (D) Cells were transfected with siRNAs as in panel B but then analysed by staining for junctional proteins. Note, GEF-H1 depleted cells still form intact monolayers. (E, F) NFkB activity was assayed by luciferase reporter gene assay in GEF-H1-depleted cells (E) or in cells transfected with the C-terminal domain (CTD) of GEF-H1 (F). The CTD of GEF-H1 blocks proinflammatory signalling.

### Example 2—Experimental Analysis of GEF-H1 Inhibitors

Embodiments of the peptide inhibitors of the present invention were based on the C-terminal domain (CTD) of GEF-H1, which has an auto-inhibitory function (see FIG. 1) and can attenuate epithelial degeneration in an RPE disease model. Removal of the C1 domain and CTD domain of GEF-H1 leads to a constitutive active mutant (GEF-H1-CA) as the C1 domain mediates sequestration of GEF-H1 to microtubules and the CTD contains an auto-inhibitory activity. The inventors mapped this auto-inhibitory activity to a specific amino acid sequence, termed F1 within the CTD (FIG. 2) that blocks GEF-H1 function either by transfection of a recombinant protein or by fusion to a TAT sequence to render it membrane-permeable (FIGS. 4, and 5). Other CTD fragments F2 and F3 (FIG. 2) did not show auto-inhibitory activity (FIGS. 4 and 5).

Other embodiments of the peptide inhibitors of the present invention were generated by modelling of the GEF-H1/RhoA complex and designing peptides that were likely to inhibit formation of the complex. Peptides P1, P2, P3, P4 and P5 were selected for further analysis. Tests resulted in the identification of P5, a 19 aa inhibitory peptide that was retained for further analysis and refinement (FIG. 3) Two derivatives of this peptide were synthesized: TAT-P5, a variant with a TAT motif to promote entry into cells, and stapled-P5, a shortened variant with a intramolecular link to stabilise the conformation (FIG. 3B).

F1 and P5 inhibitors were then synthesized in a membrane-permeable form using TAT-motifs, a strategy used for molecules already in clinical trials. TAT-modified versions were found to effectively block GEF-H1 function in cell-based assays (see below). A scrambled version of P5 was synthesized to be used as a negative control (FIG. 3B, SEQ ID NO: 8).

In Vitro Analysis and Cell-Based Assays

The potency of GEF-H1 inhibitors was analysed in cell-based assays to determine effectiveness of inhibition of GEF-H1 signalling and specificity for GEF-H1, and in cell-based assays modelling epithelial degeneration or inflammatory disease. Potential toxicity of the inhibitors was determined by measuring apoptosis (Caspase3/7 activation) and necrosis (release of lactase dehydrogenase).

1) F1 (FIG. 2), was successfully identified by testing recombinant fragments generated from the C-terminal domain of GEF-H1 for inhibition of overexpressed GEF-H1 using morphological assays (cell spreading) and a transcriptonal SRE-driven reporter assay (FIG. 4). The same assays were used to test inhibitors predicted from the RhoA/GEF-H1 modelling (P1 to P5), leading to the identification of P5 as the most effective inhibitor (FIGS. 3 and 4).

2) Active GEF-H1 binds with high affinity to nucleotide-free and GDP-bound RhoA, its physiological substrate. Pull down assays revealed that the F1 inhibitor and the P2, P4 and P5 inhibitors inhibited the interaction between RhoA and constitutively active GEF-H1, which contains the catalytically active DH/PH module (FIG. 5). Thus, both F1 and P5 block GEF-H1 function by the same mechanism. Based on these functional and biochemical assays (FIGS. 4 and 5), F1 and P5 were selected for further analysis and refinement. Membrane permeable derivatives were synthesized for both inhibitors: TAT-F1 and TAT-P5.

The P5 sequence and modified versions thereof, are derived from the interaction surface of RhoA with the active site of GEF-H1 (i.e., the DH domain). Hence, they block binding of RhoA to a constitutively active mutant of GEF-H1 that consists of the DH/PH module as most of the N- and C-terminal domains has been deleted (FIG. 5). The same is true for the F1 sequence, as it blocks binding of RhoA to a DH/PH domain construct (FIG. 5).

3) Repetition of the morphological assays and reporter gene assays using a the alpha-smooth muscle actin (αSMA) promoter, an SRE-element containing promoter that is activated by GEF-H1 signalling in pathologial processes. Both membrane-permeable inhibitors effecitvely inhibited GEF-H1 signalling and revealed low μmolar IC50s in cell-based reporter assays (FIG. 6). FIG. 6 shows concentration-dependence of inhibition for both F1 and P5. Thus, based on the evidence set out above, both the F1 and P5 peptides function by inhibiting the binding of RhoA to GEF-H1.

4) The inventors next tested the specificity of the inhibitors using closely related GEfs. p114RhoGEF, a very closely related RhoA GEF based on homology analysis, and Dbl, the founding member of the Dbl domain GEFs. Morphological assays and reporter gene assays revealed that neither GEF was affected by the two inhibitors (FIG. 7).

5) Toxicity assays assessing cell death by apoptosis (caspase 3/7 activation) and necrosis (LDH realse) did not reveal a significant increase in cell death if two different cell lines were treated with incrasing concentrations of the two inhibitors (FIG. 8). This indicated that the two inhibitors were not cytotoxic at the concentrations required for inhibition of GEF-H1 signalling.

6) To refine the P5 inhibitor further, a stapled, shorter derivative was synthesized with an intramolecular bridge stabilising the helical confirmation. Cell based assays showed that this stapled-P5 sequence, which shares the same core seqeunce as P5, see SEQ ID NOs: 1, 13, 14, and 15, inhibited GEF-H1 signalling in overexpression assays assessing morphology and transcriptonal activity of GEF-H1-regulated promoters (FIG. 9A and B). Signalling of p114RhoGEF and Dbl was not affected (FIG. 9C and D). Stapled-P5 thus specifically inhibited GEF-H1. The cytotoxicity assays revealed that stapled-P5 was not cytotoxic at concentrations required for GEF-H1 inhibition (FIG. 10).

7) The inventors next tested the inhibitors in cell-based disease models. GEF-H1 drives fibrotic and degenrative responses in the RPE, which can be modelled by treating primary RPE cells with TGF-β. FIG. 11 shows that TAT-F1, TAT-P5 and stapled-P5 effectively suppress TGF-β-induced induction of αSMA, a marker of epithelial fibrosis, and inhibit downregulation of junctional markers, a process linked to epithelial barrier dysfunction. In retinal disease, TGF-β induces migration of RPE cells; migration of TGF-β-treated RPE cells was effectively inhibited by TAT-P5 (FIG. 12A). The inhibitor also effectively prevented the degeneration of human RPE cells generated from embryonic stem cells (FIG. 12B).

8) GEF-H1 regulates inflammatory responses in various cell types. Hence, the inventors tested the inhibititors in an endothelial inflammatory model. Cell permeable GEF-H1 inhibitors blocked lipopolysaccharide (LPS)-induced cytoskeletal remodeling and inflammatory marker ICAM1 expression, and attenuated barrier deregulation in response to LPS (FIG. 13). Similarly, cytoskeletal remodeling and cell junction disruption was also attenuated in TNFα and Thrombin-stimulated endothelial cells.

9) The inventors tested whether the inhibitors inhibited NF-κB-mediated transcription stimulated by constitutively active GEF-H1. TAT-P5 and stapled-P5 inhibited NF-κB-mediated transcription stimulated by constitutively active GEF-H1 (FIG. 14).

10) GEF-H1 stimulates RhoA signalling during tumour cell migration. Therefore, the inventors analysed whether the inhibitors impact on the migration of metastatic tumour cells. TAT-P5 and stapled-P5 strongly attenuated migration of invasive cancer cells (FIG. 15).

These data demonstrate that GEF-H1 activity and function can be blocked effectively and specifically in in vitro models, as well as in cell-based primary cell systems that model degenerative, fibrotic, and inflammatory disease.

### Example 3—In Vivo Analysis Using Mouse Disease Models

Experimental Autoimmune-Induced Uveoretinitis

The in vitro models indicated that GEF-H1 inhibitors attenuated TGFβ responses in RPE cells and proinflammatory signalling in primary endothelial cells. Therefore, the inventors tested whether inhibition of GEF-H1 could rescue experimental autoimmune-induced uveoretinitis (EAU). Factors such as TNFα play an important role in this inflammatory disease model, and in vitro data indicated that inhibiting GEF-H1 counteracts TNFα signalling. EAU was induced in mice and the GEF-H1 inhibitors were tested topically (daily, in the form of eye drops) and systemically (every two days; intraperitoneally or subcutaneously). To model more closely a real life-disease treatment, treatment was started once inflammation was detected by bright field ocular microscopy (fundus imaging). Ocular inflammation in mice normally starts at day 10-14. The inventors followed disease progression with a topical endoscopic fundus imaging system for 2-3 weeks after treatment started and then eyes were collected for histological analysis.

FIG. 16 shows a clinical scoring of pathological disease development once treatment started. GEF-H1 inhibitors not only inhibited disease progression but led to a significant improvement of the clinical score. The TAT-P5 inhibitor had the strongest overall effect based on fundus imaging and led to an improved outcome in the retinal structure as assessed by histology at the end of the treatment (FIG. 17).

Thus, GEF-H1 inhibitors can arrest and reverse intraocular inflammation. Importantly, animals treated systemically did not show obvious sign of ill health, indicating that the inhibitors do not have strong toxic effects.

Allergic Conjunctivitis

Allergic inflammatory disease can be modeled in mice by sensitizing animals with ovalbumin (OVA). After sensitizing, mice were challenged with OVA in the presence or absence of the TAT-P5 GEF-H1 inhibitor applied as eye drops. FIG. 18, shows that TAT-P5 inhibited disease as assessed clinically as well as histologically.

Competitive Advantage and Perspectives

GEF-H1 inhibition has the advantage of targeting disease-relevant signaling in a process-specific manner. Rho GTPases and their effectors often regulate opposing processes; hence they are not process-specific. In contrast, Rho GEFs are much more process-specific than the GTPases themselves. For example, RhoA-driven ROCK signaling drives epithelial barrier formation but also epithelial degeneration and barrier disruption. These opposing processes are activated by distinct RhoA GEFs that drive RhoA signaling in a process-specific manner. Hence, targeting GEF-H1, the GEF that drives epithelial degeneration and inflammation, enables specific inhibition of disease-relevant signaling that is essential for successful therapeutic applications.

### REFERENCES

Birukova, A. A., P. Fu, J. Xing, B. Yakubov, I. Cokic, and K. G. Birukov. 2010. Mechanotransduction by GEF-H1 as a novel mechanism of ventilator-induced vascular endothelial permeability. Am. J. Physiol. L837-L848.
Mambetsariev, I., Y. Tian, T. Wu, T. Lavoie, J. Solway, K. G. Birukov, and A. A. Birukova. 2014. Stiffness-activated GEF-H1 expression exacerbates LPS-induced lung inflammation. PLoS One. 9:e92670.
Mizuarai, S., K. Yamanaka, and H. Kotani. 2006. Mutant p53 induces the GEF-H1 oncogene, a guanine nucleotide exchange factor-H1 for RhoA, resulting in accelerated cell proliferation in tumor cells. Cancer Res. 66:6319-6326.
Sambrook et al. (1989, Molecular Cloning—a laboratory manual; Cold Spring Harbor Press).
Terry, S., M. Nie, K. Matter, and M. S. Balda. 2010. Rho signaling and tight junction functions. Physiology (Bethesda). 25:16-26.
Tsapara, A., P. Luthert, J. Greenwood, C. S. Hill, K. Matter, and M. S. Balda. 2010. The RhoA activator GEF-H1/Lfc is a transforming growth factor-beta target gene and effector that regulates alpha-smooth muscle actin expression and cell migration. Mol Biol Cell. 21:860-870.
Varma, H., A. Yamamoto, M. R. Sarantos, R. E. Hughes, and B. R. Stockwell. 2010. Mutant huntingtin alters cell fate in response to microtubule depolymerization via the GEF-H1-RhoA-ERK pathway. J Biol Chem. 285:37445-37457.

1. A peptide that blocks the function of human Guanine Nucleotide Exchange Factor-H1 (GEF-H1), wherein the peptide comprises an amino acid sequence that has at least 80% sequence identity to SEQ ID NO: 1, SEQ ID NO: 13, SEQ ID NO: 14, SEQ ID NO: 15, or SEQ ID NO: 2.

2. The peptide according to claim 1, wherein the peptide comprises an amino acid sequence that has at least 90% sequence identity to SEQ ID NO: 1, SEQ ID NO: 13, SEQ ID NO: 14, SEQ ID NO: 15, or SEQ ID NO: 2, wherein the peptide blocks the function of GEF H1.

3. The peptide according to claim 1, wherein the peptide comprises an amino acid sequence that has at least 95% sequence identity to SEQ ID NO: 1, SEQ ID NO: 13, SEQ ID NO: 14, SEQ ID NO: 15, or SEQ ID NO: 2, wherein the peptide blocks the function of GEF H1.

4. The peptide according to claim 1, wherein the peptide comprises the sequence of SEQ ID NO: 1, SEQ ID NO: 13, SEQ ID NO: 14, SEQ ID NO: 15, SEQ ID NO: 3, SEQ ID NO: 2, or SEQ ID NO: 6.

5. The peptide according to claim 1, wherein the peptide comprises:
a) the sequence of SEQ ID NO: 1, SEQ ID NO: 13, SEQ ID NO: 14, SEQ ID NO: 15, SEQ ID NO: 2, or SEQ ID NO: 3; and
b) the sequence of SEQ ID NO: 4.

6. The peptide according to claim 1, wherein the peptide comprises SEQ ID NO: 7 or SEQ ID NO: 5.

7. The peptide according to claim 1, wherein the peptide has the sequence of SEQ ID NO: 1, SEQ ID NO: 13, SEQ ID NO: 14, SEQ ID NO: 15, SEQ ID NO: 3, SEQ ID NO: 5, SEQ ID NO: 6, SEQ ID NO: 2, or SEQ ID NO: 7.

8. The peptide according to claim 1, wherein the peptide is bound or conjugated to an additional moiety.

9. A polynucleotide sequence encoding the peptide according to claim 1.

10. A vector comprising the polynucleotide of claim 9.

11. The vector according to claim 10, which is a viral vector.

12. A pharmaceutical composition comprising the peptide according to claim 1, or a vector comprising a polynucleotide encoding the peptide, and a pharmaceutically acceptable carrier.

13-18. (canceled)

19. A method of treatment of disease comprising administering a therapeutically effective amount of the peptide according to claim 1, a vector comprising a polynucleotide encoding the peptide, or a pharmaceutical composition comprising (a) the peptide or the vector and (b) a pharmaceutically acceptable carrier.

20. A method of treating an inflammatory or degenerative disease, or cancer, comprising administering a therapeutically effective amount of the peptide according to claim 1, a vector comprising a polynucleotide encoding the peptide, or a pharmaceutical composition comprising (a) the peptide or the vector and (b) a pharmaceutically acceptable carrier, to a patient in need thereof.

21. (canceled)

22. A kit comprising the peptide according to claim 1, a vector comprising a polynucleotide encoding the peptide, or a pharmaceutical composition comprising (a) the peptide or the vector and (b) a pharmaceutically acceptable carrier, and instructions for use.

23. The method of claim 20, wherein the inflammatory or degenerative disease is an allergic and inflammatory disease of the eye or other organ such as the lung; an autoimmune disease; a fibrotic disease of the eye or lung, kidney or liver; diabetes; a neurological degenerative disease; or cancer.

24. The method of claim 23, wherein the disease to be treated is corneal inflammation, conjunctivitis, asthma, chronic obstructive pulmonary disease, uveoretinitis, multiple sclerosis, rheumatoid arthritis, proliferative vitreoretinopathy, choroidal neovascularisation fibrosis, glaucoma, trabecular meshwork fibrosis, idiopathic pulmonary fibrosis, diabetic retinopathy, diabetic nephropathy, Huntington disease, Alzheimer's disease or metastatic cancer.

25. The method of claim 23, wherein the peptide, vector or pharmaceutical composition is administered via a parenteral route of administration such as an intravenous, subcutaneous, intraocular, intramuscular, intradermal, intraperitoneal, spinal route or by injection or infusion; or by another administration route such as rectal, oral, ocular, topical, epidermal, mucosal, local, peritumoral, juxtatumoral, intratumoral, to the resection margin of tumors, intralesional, perilesional, by intra cavity infusion, intravesicle administration, or by inhalation.

26. The method of claim 23, wherein the peptide, vector or pharmaceutical composition is administered in combination with an additional therapeutic agent.
